(19) United States
(12) Patent Application Publication
(10) Pub. No.: US 2020/0009295 A1
(43) Pub. Date: Jan. 9, 2020

(54) Functionalized Scaffold To Promote Meniscus Repair

(71) Applicant: The Scripps Research Institute, La Jolla, CA (US)

(72) Inventors: Martin Lotz, La Jolla, CA (US); Kwang Il Lee, San Diego, CA (US)

(21) Appl. No.: 16/494,746

(22) PCT Filed: Mar. 16, 2018
(86) PCT No.: PCT/US2018/023015

§ 371 Date: Sep. 16, 2019

Related U.S. Application Data
(60) Provisional application No. 62/472,917, filed on Mar. 17, 2017.

(51) Int. Cl. A61L 27/36; A61K 31/727; A61K 38/18; A61L 27/38; A61L 27/56; A61L 27/24; A61L 27/54

(52) U.S. Cl. CPC A61L 27/3654; A61L 27/3612; A61K 31/727; A61K 38/1858; A61L 27/3834; A61L 27/3817; A61L 2430/06; A61L 27/24; A61L 27/54; A61L 2300/252; A61L 2300/236; A61L 2300/414; A61L 27/56

(57) ABSTRACT

Provided herein is a scaffold comprising a decellularized meniscus tissue, wherein the scaffold is covalently conjugated with heparin and a growth factor. Also provided herein is a method of repairing and/or treating a tissue injury in a subject in need thereof, comprising: providing a scaffold comprising a decellularized meniscus tissue; and repairing and/or treating the tissue injury by implanting the scaffold over the tear, wherein the scaffold is covalently conjugated with heparin and a growth factor.

### CROSS-REFERENCE TO RELATED APPLICATIONS

This application claims the benefit of priority to U.S. Provisional Patent Application Ser. No. 62/472,917 filed Mar. 17, 2017, the entire contents of each of which is incorporated herein by reference.

### STATEMENT REGARDING FEDERALLY SPONSORED RESEARCH

The invention was made with government support under Grant No. AG007996 awarded by the National Institutes of Health (NIH). The government has certain rights in the invention.

### FIELD OF THE INVENTION

The present disclosure relates to scaffolds and methods of tissue repair and/or regeneration using the scaffolds.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Meniscus tears are among the most common knee injuries. Tears occur because of forceful twisting, rotating, or hyper-flexing of the knee joint. A torn meniscus causes knee pain, swelling, stiffness, and limitations in extending the knee. Meniscal tears, in particular, the most prevalent forms that occur in the inner third, typically do not spontaneously heal and represent a major risk factor for the development of knee osteoarthritis (OA). Strategies for meniscal repair are thus essential to prevent disability and pain associated with OA.

Although several treatments currently exist for meniscal injuries, the treatment options do not result in meniscal repair or regeneration. The majority of meniscal injuries are treated by a partial meniscectomy. While patients might respond well to this treatment in the short term, they often develop OA several years postoperatively. The amount of tissue removed has been linked to the extent and speed of cartilage degeneration. When the majority of the meniscal tissue is affected by the injury, a total meniscectomy is performed. If the patient experiences pain after a total meniscectomy without significant joint degeneration, a secondary treatment with meniscal allografts is possible. However, the use of allografts is limited by tissue availability and by narrow indications.

Meniscal repair and regeneration is mediated through the migration and proliferation of fibroblasts that originate from the adjacent synovium and joint capsule. These cells produce a fibrovascular scar tissue, which under appropriate environmental conditions, such as oxygen concentration and hydrostatic pressure, undergoes a process of fibrocartilaginous metaplasia resulting in a modification of the fibrous tissue into fibrocartilage. Fibroblasts will not synthesize fibrocartilaginous tissue de novo. Thus, the external environmental stimuli are required to modulate the fibrous connective tissue into fibrocartilage. Accordingly, there remains a need for novel tissue repair devices capable of encouraging meniscal tissue regeneration, as well as, methods for using such tissue repair devices.

### SUMMARY OF THE INVENTION

Various embodiments disclosed herein include a scaffold comprising: a decellularized meniscus tissue, wherein the scaffold is covalently conjugated with heparin and a growth factor. In one embodiment, the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1. In one embodiment, the growth factor is platelet derived growth factor (PDGF). In one embodiment, the PDGF is PDGF-AA, PDGF-BB, and/or PDGF-AB. In one embodiment, the scaffold further comprises stem cells. In one embodiment, the scaffold further comprises meniscus cells. In one embodiment, the decellularized meniscus tissue comprises collagen fibers, and wherein the collagen fiber orientation is matched with that of a meniscus defect. In one embodiment, the decellularized meniscus tissue comprises pores. In one embodiment, the pores are created in the decellularized meniscus tissue by collagenase digestion, mechanical puncture, and/or laser application. In one embodiment, the scaffold releases the growth factor with substantially first order kinetics over a period of at least 10 days after administration. In one embodiment, the scaffold releases the growth factor with substantially first order kinetics over a period of at least 20 days after administration. In one embodiment, the scaffold releases the growth factor with substantially first order kinetics over a period of at least 30 days after administration. In one embodiment, the tensile strength of the scaffold is at least two times greater than a tensile strength of a similar decellularized meniscus tissue, but without covalent conjugation of heparin and the growth factor. In one embodiment, the tensile strength of the scaffold is at least three times greater than a tensile strength of a similar decellularized meniscus tissue, but without covalent conjugation of heparin and the growth factor. In one embodiment, the tensile modulus of the scaffold is greater than 0.6 Young's Modulus (MPa). In one embodiment, the growth factor comprises between 10 ng/mL to 1 mg/mL of the scaffold. In one embodiment, the decellularized meniscus tissue is essentially in a sheet form. In one embodiment, the decellularized meniscus tissue has a three dimensional form. In one embodiment, the scaffold is in a medical dressing. In one embodiment, the decellularized meniscus tissue originates from a mammal. In one embodiment, the decellularized meniscus tissue originates from a human. In one embodiment, the scaffold is in a sterile condition and packaged in a sterile container. Various embodiments disclosed herein also include a method of repairing and/or treating a tissue injury in a subject in need thereof, comprising: providing a scaffold comprising a decellularized meniscus tissue; and repairing and/or treating the tissue injury by implanting the scaffold over the tear, wherein the scaffold is covalently conjugated with heparin and a growth factor. In one embodiment, the tissue injury is a tear in the tissue. In one embodiment, the tissue is a meniscus tissue. In one embodiment, the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1. In one embodiment, the growth factor is platelet derived growth factor (PDGF). In one embodiment, the PDGF is PDGF-AA, PDGF-BB, and/or PDGF-AB. In one embodiment, the scaffold recruits new population of cells to initiate repair in the avascular or vascular zone of meniscus tissue. In one embodiment, the scaffold is optimized for effective cell infiltration and migration from host cells to the scaffold. In one embodiment, the acellular scaffold is implanted over the meniscus tear by an arthroscopic surgery. In one embodiment, the scaffold releases the growth factor with substantially first order kinetics over a period of at least 10 days after administration. In one embodiment, the scaffold releases the growth factor with substantially first order kinetics over a period of at least 20 days after administration. In one embodiment, the scaffold releases the growth factor with substantially first order kinetics over a period of at least 30 days after administration. In one embodiment, the tensile strength of the scaffold is at least two times greater than a tensile strength of a similar decellularized meniscus tissue, but without covalent conjugation of heparin and PDGF. In one embodiment, the tensile strength of the scaffold is at least three times greater than a tensile strength of a similar decellularized meniscus tissue, but without covalent conjugation of heparin and PDGF. In one embodiment, the tensile modulus of the scaffold is greater than 0.6 Young's Modulus (MPa). In one embodiment, PDGF comprises between 10 ng/ml to 1 mg/ml of the scaffold. In one embodiment, the method of repairing and/or treating the tear in the tissue further comprises a second treatment regimen. In one embodiment, the second treatment regimen comprises a non-surgical treatment, such as rest, ice, compression, elevation, and/or physical therapy. In one embodiment, the second treatment regimen comprises a surgical treatment such as surgical repair, partial meniscectomy, and/or total meniscectomy. In one embodiment, the subject is a mammal. In one embodiment, the subject is a human. In one embodiment, the subject is a horse.

Further embodiments of the present disclosure include a kit comprising: a sterile container comprising a scaffold covalently conjugated with heparin and a growth factor; and instructions for using the kit. In one embodiment, the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1. In one embodiment, the growth factor is platelet derived growth factor (PDGF). In one embodiment, the PDGF is PDGF-AA, PDGF-BB, and/or PDGF-AB. In one embodiment, the kit further comprises a means for delivery of the scaffold into an injured meniscus. In one embodiment, the means of delivery is medical glue, medical sutures, medical staples, and/or medical anchors. In one embodiment, the scaffold is a biological acellular scaffold. In one embodiment, the scaffold is derived from decellularized native meniscus tissue. In one embodiment, the acellular scaffold recruits new population of cells to initiate repair in the avascular or vascular zone. In one embodiment, the heparin conjugation enables slow release of the growth factor. In one embodiment, the slow release occurs over a period of up to 30 days.

Embodiments of the present disclosure also include a device comprising: an acellular scaffold covalently conjugated with heparin and a growth factor, wherein the device is for repairing tissues. In one embodiment, the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1. In one embodiment, the growth factor is platelet derived growth factor (PDGF). In one embodiment, the acellular scaffold is a biological acellular scaffold. In one embodiment, the acellular scaffold is derived from decellularized native meniscus tissue. In one embodiment, the acellular scaffold has similar biological and mechanical characteristics compared with native meniscus. In one embodiment, the growth factor recruits new population of cells to initiate repair in the avascular zone. In one embodiment, the acellular scaffold is optimized for effective cell infiltration and migration from host cells to the scaffold. In one embodiment, the device enables slow release of the growth factor. In one embodiment, the slow release occurs over a period of up to 30 days.

Embodiments of the present disclosure further include a method of inducing cell migration, comprising: providing a decellularized meniscus scaffold for the immobilization of one or more growth factors; and inducing cell migration to the decellularized meniscus scaffold. In one embodiment, the one or more growth factors is PDGF. In one embodiment, heparin is used for immobilization. In one embodiment, the decellularized meniscus scaffold is implanted directly to a subject. In one embodiment, the subject is a mammal. In one embodiment, the subject is human.

Other features and advantages of the invention will become apparent from the following detailed description, taken in conjunction with the accompanying drawings, which illustrate, by way of example, various embodiments of the invention.

### DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with embodiments herein, a method for fibrochondrogenic differentiation during healing of meniscus tears

FIG. 2 depicts, in accordance with embodiments herein, novel technique of integrative healing by cell recruitment.

FIG. 3 depicts, in accordance with embodiments herein, decellularized meniscus scaffold (DMS) from bovine meniscus

FIG. 4 depicts, in accordance with embodiments herein, schematic diagram of PDGF-BB immobilization on heparin conjugated DMS.

FIG. 5 depicts, in accordance with embodiments herein, decellularization and PDGF conjugation of bovine meniscus. (A) image of decellularized meniscus blocks; (B) DNA content; (C) image of Toluidine Blue stained DMS; (D) quantification of Toluidine Blue content.

FIG. 6 depicts, in accordance with embodiments herein, PDGF-BB release kinetics from DMS. PDGF-BB was conjugated to DMS or heparin coated DMS and DMS was cultured at 37° C. for up to 16 days. Each type of DMS was conjugated with 100 ng of PDGF-BB. Supernatants were collected at the indicated time points and analyzed for PDGF-BB by ELISA. Results are from 3 separate experiments.

FIG. 7 depicts, in accordance with embodiments herein, anti PDGFRβ immunohistochemistry of meniscus specimen: (A) & (B) Human meniscus; (C) & (D) DMS inserted into bovine meniscus explants after 2 weeks ex-vivo culture, DMS was conjugated with heparin and 50 ng/ml PDGF-BB; (E) anti PDGFRβ positive cells (%) from (C) & (D).

FIG. 8 depicts, in accordance with embodiments herein, comparative DAPI images of (A) native bovine meniscus; (B) DMS inserted into meniscus explant; (C) PDGF coated DMS inserted into meniscus explant. Staining images of PDGF-BB (50 ng/ml) coated DMS inserted into bovine explants after 2 weeks culture; (D) DAPI; (E) Safranin-O; (F) Picrosirius red. Black arrows indicate newly produced ECM.

FIG. 9 depicts, in accordance with embodiments herein, DMS with heparin and PDGF-BB (200 ng/ml) conjugation after 2 weeks culture. (A) DAPI stain; (B) Safranin-O stain; (C) polarized light view of picrosirius red stain.

FIG. 10 depicts, in accordance with embodiments herein, tensile test after ex vivo culture at 2 and 4 weeks

FIG. 11 depicts, in accordance with embodiments herein, growth factor immobilization by heparin conjugation.

FIG. 12 depicts, in accordance with embodiments herein, ex vivo models.

FIG. 13 depicts, in accordance with embodiments herein, mechanical test after ex-vivo culture.

FIG. 14 depicts, in accordance with embodiments herein, anti PDGFRβ in human and bovine meniscus (2 weeks).

FIG. 15 depicts, in accordance with embodiments herein, cell migration in injured meniscus explants cultured with inserted DMS. DAPI stained sections of explants cultured for 2 weeks (n=3-6 per group, 40×). (a.) Native non injured meniscus; (b.) Injured meniscus cultured without DMS; (c.) Injured meniscus cultured with DMS; (d.) Injured meniscus cultured with PDGF-DMS; (e.) Graph with numbers of migrated cells. Data represent the mean of 6-8 values from 3 separate experiments.

FIG. 16 depicts, in accordance with embodiments herein, safranin-O and picrosirius red stain analysis (2 & 4 weeks).

FIG. 17 depicts, in accordance with embodiments herein, mechanical properties of the injured meniscus explants cultured with DMS. Injured explants were inserted with DMS or PDGF-conjugated DMS and cultured for 2 and 4 weeks. Tensile properties were measured by pulling to failure. Data represent the mean of 7-10 values from 3 separate experiments.

FIG. 18 depicts, in accordance with embodiments herein, ex vivo culture with DMS or PDGF conjugated DMS. (A) DAPI staining and (B) quantitative analysis, (C) anti-PDGFRβ IHC and (D) quantitative analysis, (E) histology images after 2 weeks, (F) tensile test after 2 and 4 weeks.

FIG. 19 depicts, in accordance with embodiments herein, preparation of PDGF-HEP-DMS: The DMS was made from decellularized bovine meniscus. After 0.1% (wt/v) heparin conjugation with DMS, PDGF was bound to heparin conjugated DMS.

FIG. 20 depicts, in accordance with embodiments herein, The quantitative analysis of anti-PDGFRβ positive cells: the positive cells were percentage by total cell number within the bovine meniscus explant. DMS inserted bovine meniscus explant (A); 50 ng/ml PDGF-BB coated DMS inserted bovine meniscus explant (B); 100 ng/ml PDGF-BB coated DMS inserted bovine meniscus explant (C); 200 ng/ml PDGF-BB coated DMS inserted bovine meniscus explant (D) after 2 week ex vivo culture.

FIG. 21 depicts, in accordance with embodiments herein, Safranin-O and Picrosirius red staining of DMS inserted bovine meniscus explants after 2 week ex vivo culture: 50 ng/ml PDGF-BB coated DMS inserted explant (A, B); 100 ng/ml PDGF-BB coated DMS inserted explant (C, D); 200 ng/ml PDGF-BB coated DMS inserted explant.

FIG. 22 depicts, in accordance with embodiments herein, Immunohistochemistry of DMS and PDGF coated DMS inserted bovine meniscus explant after 2 week ex vivo culture: anti-Aggrecan (A); anti-Collagen type 1a1 (B); anti-MKX (C); anti-Collagen type 2a1 (D).

FIG. 23 depicts, in accordance with embodiments herein, PDGFRβ positive cells in injured meniscus explants. Anti-PDGFRβ stained sections of explants cultured for 2 weeks (n=3-6 per group, 40×). (a.) Native non injured meniscus; (b.) Injured meniscus cultured without DMS; (c.) Injured meniscus cultured with DMS; (d.) Injured meniscus cultured with PDGF-DMS; (e.) Graph with numbers of migrated cells. Data represent the mean of 6-8 values from 3 separate experiments.

FIG. 24 depicts, in accordance with embodiments herein, ECM formation in the injured meniscus explants. (a-b.) Safranin-O staining: Native non-injured meniscus for 2 and 4 weeks; (c-d.) Safranin-O staining: Injured meniscus without DMS for 2 and 4 weeks; (e-f) Safranin-O staining: Injured meniscus cultured with DMS for 2 and 4 weeks; (g-h.) Safranin-O staining: Injured meniscus cultured with PDGF-DMS for 2 and 4 weeks; (i-j.) Picrosirius red staining: Injured meniscus cultured with DMS for 2 and 4 weeks; (k-l.) Picrosirius red staining: Injured meniscus cultured with PDGF-DMS for 2 and 4 weeks; (m-n.) Safranin-O positive stained area (% of total area) and integration % between DMS and explant assessed by pircrosirius red staining and shown as % integrated interface of total interface area.

FIG. 25 depicts, in accordance with embodiments herein, alignment of the collagen fiber orientation of the DMS with the collagen fiber orientation in the meniscus defect. Tissues were harvested in defined orientation (a) AVAS_Section from vertically punctured cylinder; (b) AVAS_Section from horizontally punctured cylinder; (c) VAS_Section from vertically punctured cylinder; and (d) VAS_Section from horizontally punctured cylinder.

FIG. 26 depicts, in accordance with embodiments herein, collagenase digestion of DMS. In one embodiment, collagenase digestion facilitated cell migration and infiltration.

FIG. 27 depicts, in accordance with embodiments herein, that PDGF-conjugated DMS induces cell migration and proliferation.

FIG. 28 depicts, in accordance with embodiments herein, that PDGF-conjugated DMS induces cell migration and proliferation.

### DETAILED DESCRIPTION

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

Meniscus tears are the most common injury of the knee joint. Meniscal tears, in particular the most prevalent forms that occur in the inner third, typically do not spontaneously heal and represent a major risk factor for the development of knee osteoarthritis.

As described herein, and in accordance with the various embodiments herein, the inventors have developed a novel chemotactic-acellular meniscus graft for integrative meniscus healing. The inventors have characterized the decellularized meniscus scaffold (DMS) for host cell infiltration; examined the effect of PDGF coating of DMS on cell recruitment and meniscus repair in vitro; and tested PDGF-coated DMS for efficacy in meniscus integrative healing using an animal model.

In one embodiment, the inventors have shown that only chemotactic growth factor could be applied to the scaffold without any exogenous cells and that the endogenous cells expected to migrate to the injured area perform the healing process. In one embodiment, the inventors have found that PDGF has strong chemotactic effect for progenitor cells. In one embodiment, the inventors have shown that PDGF application with decellularized meniscus as a scaffold is able to heal meniscus injury by endogenous cell migration.

In various embodiments, the inventors have shown that heparin conjugated decellularized meniscus scaffolds binds and slowly releases PDGF-BB over at least two weeks. In another embodiment, the inventors have shown that insertion of the PDGF treated scaffold in defects in avascular meniscus led to increased PDGFRβ expression and cell migration into the defect zone. In another embodiment, safranin-O and picrosirius red staining showed tissue integration between the scaffold and injured explants. In another embodiment, tensile properties of injured explant treated with PDGF coated scaffold were significantly higher than in the scaffold without PDGF.

In one embodiment, disclosed herein is a scaffold, comprising a decellularized meniscus tissue, wherein the scaffold is covalently conjugated with heparin and a growth factor. In one embodiment, the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1. In one embodiment, the growth factor is platelet derived growth factor (PDGF). In one embodiment, the PDGF is PDGF-AA, PDGF-BB, and/or PDGF-AB. In one embodiment, the scaffold further comprises stem cells. In one embodiment, the scaffold further comprises meniscus cells. In one embodiment, the decellularized meniscus tissue comprises collagen fibers, and wherein the collagen fiber orientation is matched with that of a meniscus defect. In one embodiment, the decellularized meniscus tissue comprises pores. In one embodiment, the pores are created in the decellularized meniscus tissue by collagenase digestion, mechanical puncture, and/or laser application. In one embodiment, the scaffold releases the growth factor with substantially first order kinetics over a period of at least 10 days after administration, or at least 20 days after administration, or at least 30 days after administration. In one embodiment, the scaffold releases the growth factor with substantially first order kinetics over a period of at least 1, 2, 4, 6, 8, 10, 12, 14, 16, 18, 20, 22, 24, 26, 28, or 30 days. In one embodiment, the tensile strength of the scaffold is at least two times greater than a tensile strength of a similar decellularized meniscus tissue, but without covalent conjugation of heparin and the growth factor. In one embodiment, the tensile strength of the scaffold is at least three times greater than a tensile strength of a similar decellularized meniscus tissue, but without covalent conjugation of heparin and the growth factor. In one embodiment, the tensile modulus of the scaffold is greater than 0.6 Young's Modulus (MPa). In one embodiment, the growth factor comprises between 10 ng/mL to 1 mg/mL of the scaffold. In one embodiment, the growth factor comprises between 1 ng/mL to 1 μg/mL, or between 1 μg/mL to 500 μg/mL, or between 500 μl g/mL to 1 mg/mL, or between 1 mg/mL to 10 mg/mL. In one embodiment, the decellularized meniscus tissue is essentially in a sheet form. In one embodiment, the decellularized meniscus tissue has a three dimensional form. In one embodiment, the scaffold is in a medical dressing. In one embodiment, the decellularized meniscus tissue originates from a mammal. In one embodiment, the decellularized meniscus tissue originates from a human. In one embodiment, the scaffold is in a sterile condition and packaged in a sterile container.

In one embodiment, the growth factor conjugated to DMS can be PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1. In one embodiment, the PDGF is PDGF-AA, PDGF-BB, and/or PDGF-AB. In one embodiment, the applied growth factor dosing is between 10 ng/ml to 1 mg/ml. In one embodiment, the origin of the scaffold can be from meniscus tissue. The meniscus can originate from human or other mammals. In one embodiment, a 3-dimensional form of DMS can be prepared to fill larger meniscus defects. The decellularization process and Heparin/PDGF conjugation are similar as that described herein for the DMS sheet. In one embodiment, the PDGF-conjugated scaffolds can also be used to attach stem cells or meniscus cells (native or modified by preculture in growth factors or viral gene transfer), for implantation into meniscus defects. In one embodiment, the Heparin/PDFG conjugated DMS will be inserted into the meniscus defect during arthroscopic surgery. The DMS is fixed by application of glue, sutures, staples or anchors. In one embodiment, the scaffold may be additionally modified to facilitate the treatment of tissue injury. For example, in one embodiment, to facilitate cell migration, the collagen fiber orientation of the DMS is matched with that of the meniscus defect. This is accomplished by cutting the DMS from the meniscus horizontally and inserting the heparin/PDGF conjugated DMS in the same orientation. In another embodiment, to facilitate cell migration and infiltration, pores are created in the dense collagen fiber network of the DMS by using collagenase digestion, mechanical puncture or laser application. In one embodiment, disclosed herein is a method of repairing and/or treating a tissue injury in a subject in need thereof, comprising: providing a scaffold comprising a decellularized meniscus tissue; and repairing and/or treating the tissue injury by implanting the scaffold over the tear, wherein the scaffold is covalently conjugated with heparin and a growth factor. In one embodiment, the tissue injury is a tear in the tissue. In one embodiment, the tissue is a meniscus tissue. In one embodiment, the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1. In one embodiment, the growth factor is platelet derived growth factor (PDGF). In one embodiment, the PDGF is PDGF-AA, PDGF-BB, and/or PDGF-AB. In one embodiment, the scaffold recruits new population of cells to initiate repair in the avascular or vascular zone of meniscus tissue. In one embodiment, the scaffold is optimized for effective cell infiltration and migration from host cells to the scaffold. In one embodiment, the acellular scaffold is implanted over the meniscus tear by an arthroscopic surgery. In one embodiment, the scaffold releases the growth factor with substantially first order kinetics over a period of at least 10 days after administration. In one embodiment, the scaffold releases the growth factor with substantially first order kinetics over a period of at least 20 days after administration. In one embodiment, the scaffold releases the growth factor with substantially first order kinetics over a period of at least 30 days after administration. In one embodiment, the tensile strength of the scaffold is at least two times greater than a tensile strength of a similar decellularized meniscus tissue, but without covalent conjugation of heparin and PDGF. In one embodiment, the tensile strength of the scaffold is at least three times greater than a tensile strength of a similar decellularized meniscus tissue, but without covalent conjugation of heparin and PDGF. In one embodiment, the tensile modulus of the scaffold is greater than 0.6 Young's Modulus (MPa). In one embodiment, PDGF comprises between 10 ng/ml to 1 mg/ml of the scaffold. In one embodiment, the method of repairing and/or treating the tear in the tissue further comprises a second treatment regimen. In one embodiment, the second treatment regimen comprises a non-surgical treatment, such as rest, ice, compression, elevation, and/or physical therapy. In one embodiment, the second treatment regimen comprises a surgical treatment such as surgical repair, partial meniscectomy, and/or total meniscectomy. In one embodiment, the subject is a mammal. In one embodiment, the subject is a human. In one embodiment, the subject is a horse.

In one embodiment, the inventors have developed a novel scaffold that can be inserted into the injured meniscus lesion to promote integrative tissue healing. Specifically, in various embodiments, a method is disclosed for preparing human decellularized meniscus (with appropriate collagen orientation); Heparin and PDGF-BB conjugation of decellularized meniscus; and a method for insertion of decellularized meniscus scaffold into torn meniscus. Bovine meniscus explants were used to create meniscus tears. Insertion of the PDGF-BB conjugated meniscus scaffold led to cell migration towards the scaffold, production of new collagenous extracellular matrix that bridged the defect and improved biomechanical properties. In one embodiment, the decellularized meniscus may be inserted into meniscus tear during arthroscopy to promote healing of meniscus lesion and prevent chronic knee pain and dysfunction.

In one embodiment, disclosed herein is a device comprising: an acellular scaffold covalently conjugated with heparin and a growth factor, wherein the device is for repairing tissues. In one embodiment, the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1. In one embodiment, the growth factor is platelet derived growth factor (PDGF). In one embodiment, the acellular scaffold is a biological acellular scaffold. In one embodiment, the acellular scaffold is derived from decellularized native meniscus tissue. In one embodiment, the acellular scaffold has similar biological and mechanical characteristics compared with native meniscus. In one embodiment, the growth factor recruits new population of cells to initiate repair in the avascular or vascular zone. In one embodiment, the acellular scaffold is optimized for effective cell infiltration and migration from host cells to the scaffold. In one embodiment, the device enables slow release of the growth factor. In one embodiment, the slow release occurs over a period of up to 30 days.

In one embodiment, disclosed herein is a method of inducing cell migration, comprising: providing a decellularized meniscus scaffold for the immobilization of one or more growth factors; and inducing cell migration to the decellularized meniscus scaffold. In one embodiment, the one or more growth factors is PDGF. In one embodiment, heparin is used for immobilization. In one embodiment, the decellularized meniscus scaffold is implanted directly to a subject. In one embodiment, the subject is human. In one embodiment, the subject is a horse.

In various embodiments, the present disclosure provides that heparin conjugated DMS showed strong immobilization of PDGF-BB, which was released slowly. PDGF-BB coated DMS promoted migration of endogenous meniscus cells to the defect area and into the scaffold. New matrix was formed that bridged the space between the native meniscus and the scaffold and this was associated with improved biomechanical properties. The PDGF-BB coated DMS is a promising approach for integrative healing of the meniscus tears.

The present disclosure is also directed to a kit comprising a scaffold. The kit is useful for practicing the inventive method of repairing and/or treating a tear in a tissue. The kit is an assemblage of materials or components, including at least one of the inventive scaffolds. Thus, in some embodiments the kit comprises a sterile container comprising a scaffold covalently conjugated with heparin and a growth factor and instructions for using the kit. In one embodiment, the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1. In one embodiment, the growth factor is platelet derived growth factor (PDGF). In one embodiment, the kit further comprises a means for delivery of the scaffold into an injured meniscus. In one embodiment, the means of delivery is medical glue, medical sutures, medical staples, and/or medical anchors. In one embodiment, the scaffold is a biological acellular scaffold. In one embodiment, the scaffold is derived from decellularized native meniscus tissue. In one embodiment, the acellular scaffold recruits new population of cells to initiate repair in the avascular zone. In one embodiment, the heparin conjugation enables slow release of the growth factor. In one embodiment, the slow release occurs over a period of up to 30 days.

The exact nature of the components configured in the inventive kit depends on its intended purpose. For example, some embodiments are configured for the purpose of treating and/or healing a tear in a tissue. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome, such as to treat, repair, and/or heal a tissue. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in the medical and bio-pharmaceutical field. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing an acellular scaffold coated with a chemotactic growth factor. The packaging material generally has an external label which indicates the contents and/or purpose of the kit and/or its components.

Embodiments of the present disclosure are further described in the following examples. The examples are merely illustrative and do not in any way limit the scope of the invention as claimed.

### EXAMPLES

### Example 1

### Generally

Forceful twisting, rotating, or hyper-flexion of the knee joint leads to traumatic tears of the meniscus. A torn meniscus causes knee pain, swelling, stiffness, and limitations in extending the knee. Current surgical approaches to address meniscus tears include suturing, and partial meniscectomy. However meniscal tears in the inner third, the avascular region, typically do not heal spontaneously or after surgical interventions and represent a major risk factor for the development of knee osteoarthritis (OA). The middle and inner zones of meniscus lack blood supply therefore have the least potential for healing.

Cells that have potential to promote meniscal repair and regeneration are present in the meniscus adjacent to the tear and in synovium and joint capsule. Application of chemotactic factors to the tear site thus has potential to recruit cells that mediate repair. Growth factors have potential to promote meniscus healing and various approaches including gene transfer into meniscus cells or application of gene transduced cells have been pursued. PDGF is a candidate for meniscus repair as it has strong chemotactic activity for chondrocytes and mesenchymal stem cells. PDGF enhances meniscal cell activity and its expression is decreased in lesions in the avascular zone. Specifically, PDGF-BB is known as the strongest mitogen and it auto-phosphorylates the PDGFRβ. This receptor is involved in cell-matrix interactions for targeted manipulation of cell growth.

In one embodiment of the present disclosure, the surface immobilization of heparin by covalent linkage or electrostatic interactions was approached to overcome the initially burst released growth factor from the scaffolds. Heparin has strong binding affinity for a various growth factors such as basic fibroblast growth factor (bFGF), transforming growth factor-β (TGF-β), vascular endothelial growth factor (VEGF), and platelet-derived growth factor-BB (PDGF-BB). In one embodiment, insertion of PDGF conjugated scaffolds by heparin immobilization into the meniscus tear region recruits cells that mediate the meniscus tear. In one embodiment, the present disclosure used decellularized meniscus as a clinically applicable scaffold for PDGF immobilization and tested its ability to recruit endogenous cells to mediate repair of the meniscus tears.

In another embodiment, PDGF and other growth factors can be immobilized on scaffolds via covalent linkage to heparin or electrostatic interactions, which in turn leads to its sustained release. In one embodiment of the present disclosure, the inventors used heparin-conjugated decellularized meniscus as a readily available and clinically applicable scaffold for PDGF-BB immobilization and showed the ability of this scaffold to recruit endogenous cells to mediate repair of the meniscus tears.

### Example 2

### Acellular Meniscus Graft

As known to a skilled artisan in the art, meniscus tears are among the most common knee injuries; and while certain treatments currently exist for meniscal injuries, the treatment options do not result in meniscal repair or regeneration. In some cases, a partial meniscectomy or total meniscectomy is performed to treat the patient. These treatments often result in osteoarthritis in the patient, and/or scar tissues.

In various embodiments, the inventors solved this problem by developing a chemotactic-acellular meniscus graft for integrative meniscus healing. In one embodiment, the inventors characterized the decellularized meniscus scaffold (DMS) for host cell infiltration. Decellularized native meniscus tissue is a promising biological scaffold for repair of the injured human meniscus. It has similar biological and mechanical characteristics compared with native meniscus. Moreover, the ideal scaffolding should also include a chemotactic factor to recruit a new population of cells to initiate repair in the avascular zone. The DMS structure was modified for effective cell infiltration and migration from host cells to the DMS.

In one embodiment, the effect of PDGF coating of DMS on cell recruitment and meniscus repair in vitro was examined. PDGF-BB (Platelet-derived growth factor-BB) is well known as a strong chemotactic growth factor for mesenchymal cells. Using meniscus explants in an in vitro injury model, cells recruited by the growth factor to the DMS were identified using specific cell-surface markers. The number of migrated cells and newly synthesized extracellular matrix (ECM) is analyzed. Biomechanical properties are tested to determine the extent to which PDGF-BB coating improves DMS application to the injured meniscus.

In one embodiment, PDGF-coated DMS is tested for efficacy in meniscus integrative healing using an animal model. The composite is tested in a rabbit meniscus defect model. Outcomes measures may include histological and biomechanical parameters.

As described herein throughout the disclosure, the inventors sought to develop a new therapeutic approach for integrative meniscus healing by using a chemotactic growth factor coated acellular scaffold. The scaffold is examined in vitro and in vivo by using relevant outcomes measures. The growth factor enhanced natural scaffold is readily manufactured without the challenges associated with using exogenous cells and lends itself to a rapid clinical translation to address a major unmet need in enhancing repair of meniscus tears and preventing chronic pain and disability due to the development of OA.

### Example 3

### Significance

The main body of the meniscus is a fibrocartilaginous semilunar structure, which is located at the peripheral aspect of the joint between the tibia and femur. The two anterior and posterior horns are connected with the tibial plateau. The meniscus provides cushioning by transducing compression and tensile-hoop stresses which are attenuated at the tibial plateau via fibrocartilaginous enthesis.

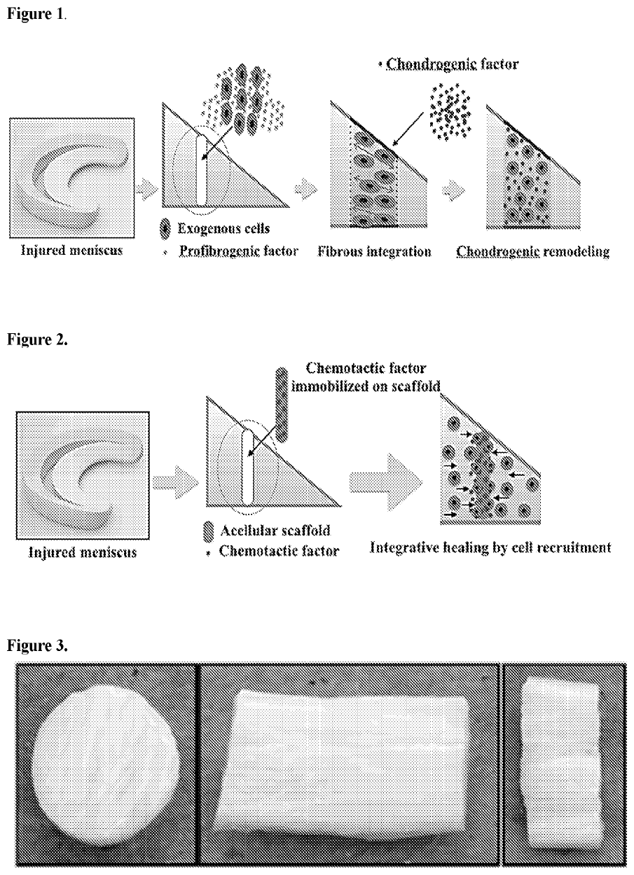

Meniscus tears are the most common injury of the knee joint. Not only normal meniscus, but also developmentally abnormal menisci, in particular discoid menisci are at increased risk for tear. Even stable discoid meniscus can present with mechanical symptoms of meniscal tear. Many cases are associated with anterior cruciate ligament (ACL) injuries such as chronic ACL insufficiency and acute ACL rupture. Different injury patterns may be associated with different risk factors such as gender, age, body weight, and injury mechanism. Even though the outer one third of the meniscus has potential healing capacity due to its vascularity and recruitment of progenitor cells from the blood, the middle and inner zones are avascular and therefore have the least potential for healing. Repair in the avascular region appears to require the proliferation of resident cells and fibrochondrocytic differentiation is essential to restoring proper meniscus biomechanical function (FIG. 1). Meniscal tears, in particular the most prevalent forms that occur in the inner third, typically do not spontaneously heal and represent a major risk factor for the development of knee osteoarthritis (OA). Strategies for meniscal repair are thus essential to prevent disability and pain associated with OA. The overall low cellularity of differentiated meniscus cells and meniscus progenitors, the dense ECM, the poor vascularity, and the inflammatory environment present at the meniscal injury site, all contribute to failure of meniscus healing and regeneration.

Due to the complex structure of the meniscus, many studies about meniscus-like differentiation using the exogenous cells were challenged (FIG. 1). In one embodiment, the application of differentiated cells, such as human meniscus cells, on electrospun scaffolds was studied. The selection of a specific growth factor varies with the specific cell type used. Different types of mesenchymal stem cells originating from bone marrow, synovium, synovial fluid, adipose tissue, meniscus, and other sources can be used in combination with growth factors to induce fibrochondrogenic differentiation. Progenitor cells when used for meniscus healing need to evolve into a heterogeneous population and concurrently synthesizing procollagens I, and IIa. Moreover, meniscus has distinct zonal characteristics, most importantly with and without vascularization.

The selection of the scaffold is also critical for adaptation to meniscus repair. Various types of scaffolds can be considered depending on the types of tears, and whether used for partial or total replacement. Even using a same raw material, scaffolds can be made of different shapes, mechanical properties, and size. Thus, many factors should be considered in scaffold design. Ideally, a scaffold for meniscus repair should have a similar fibro-cartilaginous structure to the native meniscus for stable load-transfer function.

Based on these considerations, migration of cells to the injured area and proper differentiation to produce fibrocartilaginous connective tissue appear to be key processes required for successful meniscus healing (FIG. 2). The motivation for this study is that only chemotactic growth factor can be applied to the scaffold without any exogenous cells and that the endogenous cells expected to migrate to the injured area perform the healing process. PDGF has strong chemotactic effect for progenitor cells. Thus the inventors investigated PDGF application with decellularized meniscus as a scaffold to determine the feasibility of this approach for healing of meniscus injury by endogenous cell migration.

### Example 4

### Scaffold to Treat Meniscus Tears

Scaffolds were prepared by decellularization of natural tissues as known in the field of tissue engineering. Most materials were reconstituted as hydrogels and were applied as void fillers or sponge like scaffolds after cross-linking. However, the meniscus is composed of a dense and complex collagen matrix. Moreover, multiple mechanical stresses such as compression and tensile-hoop affect the knee meniscus. In one embodiment, the meniscus derived graft was found to be a highly feasible starting material to promote healing of injured meniscus. An acellular sheet was developed from bovine meniscus for the specific application in meniscus tears (FIG. 3), where the native tissue structure was maintained with effective decellularization.

### Example 5

### Conjugation of DMS with Chemotactic Growth Factor

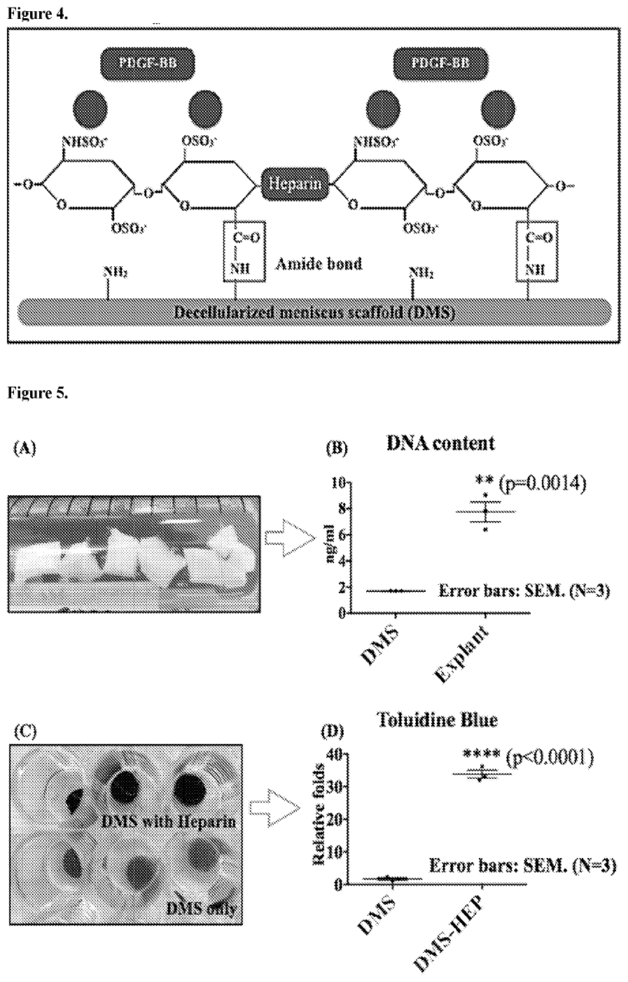

Heparin is a highly sulfated glycosaminoglycan, which possesses strong binding affinity for a various growth factors, including basic fibroblast growth factor (bFGF), transforming growth factor-β (TGF-β), vascular endothelial growth factor (VEGF), and platelet-derived growth factor-BB (PDGF-BB). The carboxyl group of heparin can bind with amine groups of DMS (FIG. 4). This composite was able to retain the growth factor and allow controlled release, which would, in a gradient, recruit resident cells from adjacent native meniscus to the injured area. Using this immobilization system, growth factor dosage and release kinetics was optimized, the phenotype of the cells recruited by the chemotactic growth factor was characterized, and the healing process after insertion of this composite into the injured meniscus was understood.

### Example 6

### Meniscus Repair and Regeneration

Tissue engineered grafts are typically composed of scaffold, exogenous cells, and growth factor. However, the use of exogenous cells is limited by challenges to harvest a sufficient amount of cells before the surgery and the complex logistics of preparing and using a live cell containing construct. The present disclosure provides a new therapeutic approach by using a chemotactic-acellular scaffold originating from native meniscus, which would be useful for the purpose of meniscus repair and regeneration. In one embodiment, one innovation of the present disclosure is in the use of a natural biomaterial, which is specifically shaped for insertion into meniscus tears and conjugated with a chemotactic growth factor to induce healing by recruiting endogenous cells. This approach also has high translational potential as it avoids challenges associated with the use of exogenous cells and uses materials and methods that are known to be safe.

### Example 7

### PDGF-BB Conjugation and Release

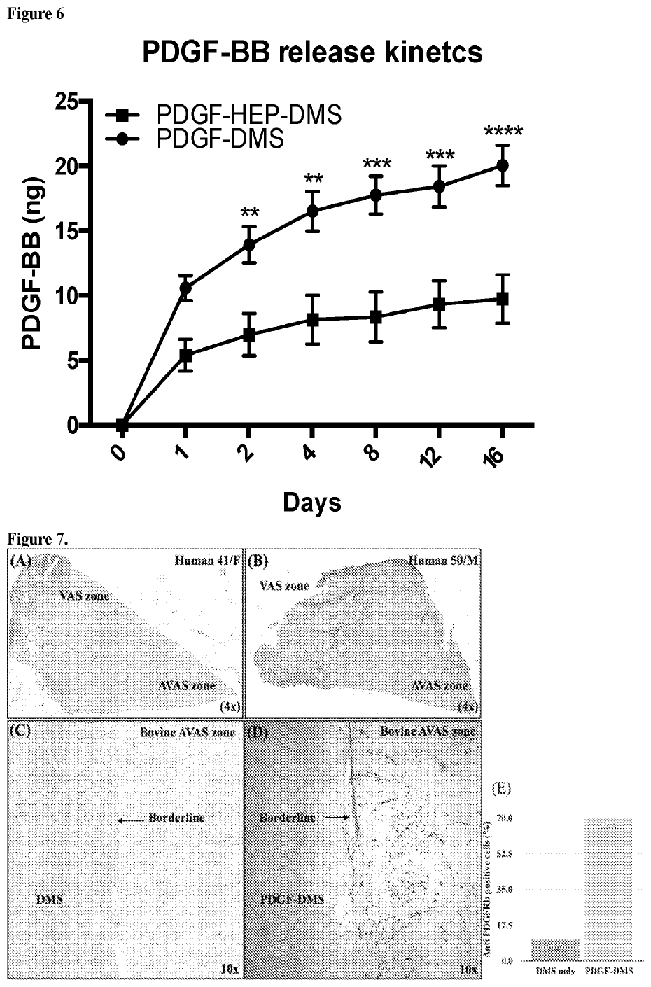

After applying the method for decellularization, DNA content was significantly decreased compared with native bovine meniscus tissue (FIG. 5 A, B). To assess efficacy of heparin conjugation, toluidine blue assay was applied for detecting heparin (FIG. 5 C). The heparin conjugation led to a large increase in toluidine blue binding (FIG. 5 D). FIG. 6 discloses experimental data on PDGF-BB conjugation and release. The amount of PDGF-BB that was bound to heparin-coated DMS was 86.72% of total 200 ng PDGF-BB and 76.82% in non heparin-coated DMS. Following an initial release of 6.22% from heparin-coated DMS versus 13.76% from non heparin-coated DMS during the first 24h, there was subsequent sustained release with about 0.61 ng per 24 h during the following 16-day period (FIG. 6). By day 16, 11.22% of the total amount of PDGF was released from heparin coated versus 26.11% release from the DMS without heparin. Also, there were significant differences in the amounts released at days 2, 4, 8, 12, and 16 between DMS with and without heparin. In one embodiment, the inventors have found that sustained release of PDGF from the heparin coated DMS continues until day 30.

PDGFRβ positive cells were found to be abundant in the vascular zone of meniscus but very rare in the avascular zone in human meniscus (FIG. 7 A, B). However, after treatment of experimental tears in bovine meniscus explants with PDGF coated scaffold, PDGFRβ positive cells were increased in the avascular zone. Moreover, the endogenous cells in the meniscus showed directed cell migration to the PDGF coated scaffold (FIG. 7 C, D).

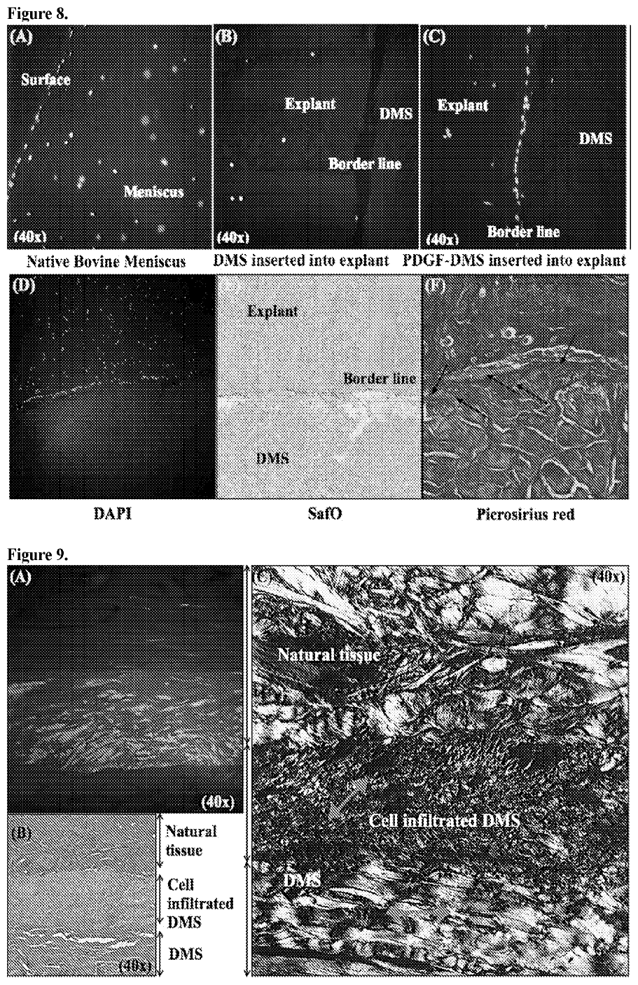

Based on the DAPI comparative images, PDGF-DMS recruited resident cells near to the borderline of the defect area compared with only DMS inserted into explant (FIG. 8 B,C). Images of safranin-O and pircrosirius red staining showed newly produced ECM from the migrated cells (FIG. 8 E, F). The cells recruited by PDGF aligned along the borderline between the meniscus tissue and the DMS (FIG. 8C). Insertion of DMS that was not coated with PDGF did not result in cell recruitment. Picosirius red staining showed that the migrated cells newly synthesized ECM which connected the DMS and native meniscus (FIG. 8F). These preliminary data suggest that PDGF is able to recruit endogenous cells to the defect area and that the cells are producing new ECM. These results illustrate that PDGF conjugated DMS has the potential to provide a chemotactic graft, which can recruit autologous cells without the requirement for any engineered exogenous cell seeding.

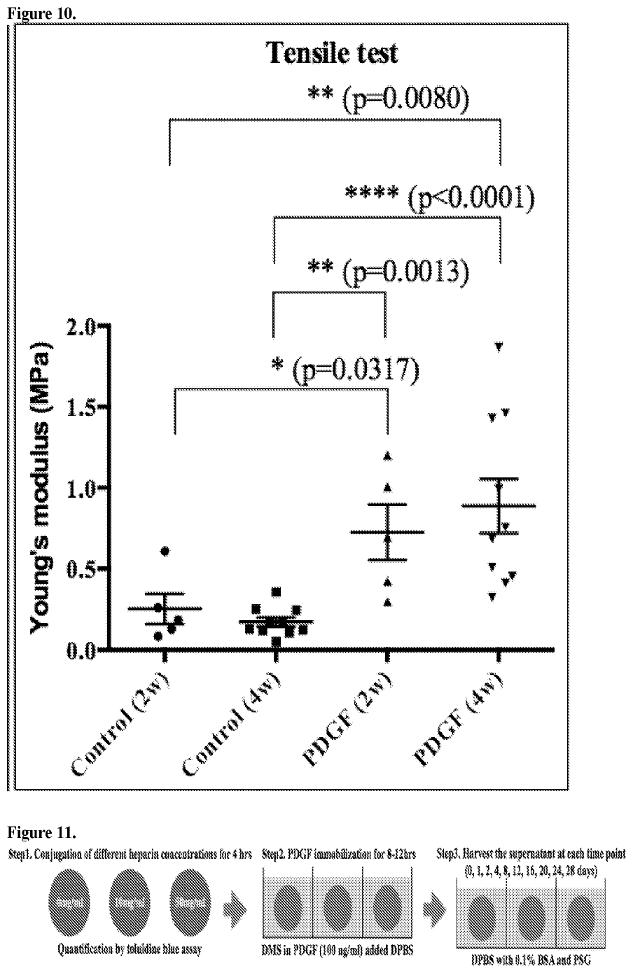

DMS coated or conjugated with 50 ng/ml PDGF showed significantly increased cell numbers recruited to the defect borderline compared to non-conjugated DMS. Even higher cell numbers were recruited with 200 ng/ml PDGF (FIG. 9.A). The newly produced ECM was filling inner space of DMS (FIG. 9.B). The 200 ng/ml group showed the directions of collagen fibers in cell infiltrated DMS layer toward to the host tissue even though the collagen fibers of DMS were horizontally aligned (FIG. 9.C). Tensile test showed significant increase in Young's modulus in PDGF groups after 2 and 4 weeks (FIG. 10).

### Example 8

### DMS Integrative Healing Approach

Optimization of the decellularized meniscus scaffold (DMS) for host cell infiltration: The DMS was optimized by surface modification for the effective cell infiltration from the host tissue to DMS, and by immobilization of chemotactic growth factor on the DMS.

The DMS has micro spaces within the scaffold due to removal of cells, proteoglycan, and GAG by the decellularization process and this may allow for attachment of recruited cells.

PDGF-BB immobilization on DMS was optimized by heparin conjugation (FIG. 11). Heparin has a PDGF binding site. The conjugated heparin on the DMS would bind PDGF-BB resulting in sustained release. Under the different heparin and PDGF concentrations, the released PDGF is quantified for up to 4 weeks by using ELISA kit (PeproTech, Inc.). Optimized result comprises over 90% binding efficacy from the initially treated PDGF-BB with sustained release lasting for 4-6 weeks.

Quantification and Characterization of Cells Migrated into the PDGF-Coated DMS:

In one embodiment, the purpose of these studies is to quantify and phenotype the migrated cells. Fresh bovine knee joints are procured and after removal of the intact menisci, explants from the avascular zone are prepared. The explants are cultured with DMEM basal media for 3 days. At that time, tear-like defects are created by using a #11 size (40 mm) scalpel and cylindrical defects are created by using a 3 mm punch (FIG. 12). The PDGF-conjugated DMS with or without PDGF is inserted into the defect area. To close the defect gap, suturing with 6-0 size nonabsorbable nylon suture was performed. The explants are incubated in DMEM basal media for 2 and 4 weeks and embedded in paraffin. The number of cells migrated to the interface between the native tissue and the DMS and the number of cells that are within the scaffold on DAPI stained sections are quantified.

ECM stains such as Trichrome, and Picrosirius red are applied after ex vivo culture to assess ECM formation and organization at the interface between native meniscus and the scaffold and even within the scaffold. The directional collagen fibers can be detected by pircrosirius red stain as well. Using the image-analysis program (ImageJ, Ver. 1.50c4), the migrated cell number and the area of newly synthesized ECM area can be quantified and statistically analyzed by Mann-Whitney, T-test (95% or 99%, confidence interval). In one embodiment, PDGF increased cellularity, organization of repair tissue, and intensity of safranin-O staining.

In one embodiment, the phenotype of the cells in terms of fibroblastic versus chondrocyte markers was determined after long-term (4 weeks) culture. Immunohistochemistry was performed for SCX, tenascin-C, collagen type 1 as fibrogenic markers and Sox9, COMP, collagen type 2 as chondrogenic markers. This analysis can address whether PDGF is not only a chemotactic factor but also promotes appropriate cell differentiation. Should the degree of chondrocytic differentiation be not sufficient, a hybrid type conjugation of DMS with PDGF and TGF-β3 may be applied.

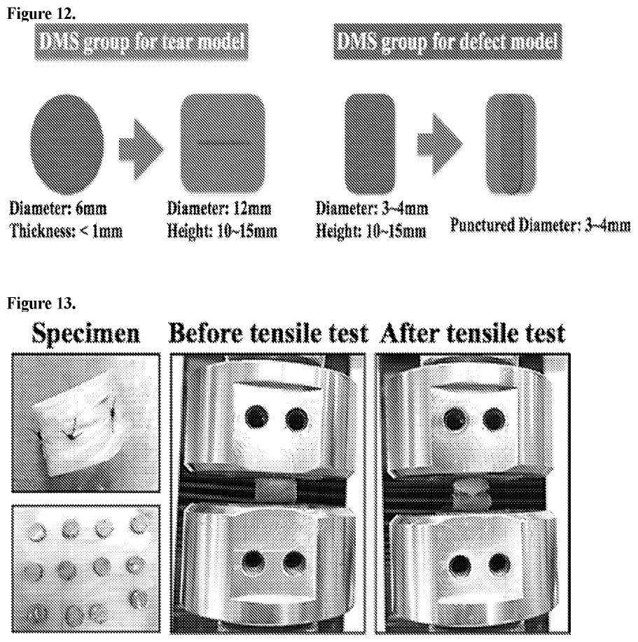

In one embodiment, biomechanical properties were examined. After making a tear of 8 mm in the middle of 12 mm diameter bovine meniscus explant, 6 mm diameter DMS with and without PDGF is inserted and sutured. After 2 and 4 weeks ex vivo culture, tensile test is performed. The specimens are fixed between the upper and lower 1,000N load cell (Instron Universal Testing Machine) using super-glue (FIG. 13). Before applying tension, the sutures are cut from the fixed explants. The tension is measured at a tension velocity of 1 mm/min. Values from each group (n=8-12) are statistically compared by Mann-Whitney, T-test (95% or 99%, confidence interval). The migrated cells may produce new ECM which may enhance the interconnectivity between the meniscus explant and the inserted DMS.

Examination of the In Vitro Optimized PDGF-Conjugated DMS for Meniscus Integrative Healing in an Animal Model:

A meniscus defect model is established in skeletally mature (2.8-3.3 kg; 4-4.5 months) female New Zealand white rabbits (n=8), following commonly used anterior lateral approach. The PDGF conjugated DMS (1 mm diameter) is inserted into the 1 mm punctured defect. To close the gaps, suturing with 6-0 size non-absorbable nylon suture is performed.

At week 4 and 8, animals are euthanized and meniscus specimens are harvested for H&E histology. Cell infiltration into the DMS and ECM synthesis from the migrated cells is analyzed. Newly produced collagens are studied by trichrome stain. For the biomechanical properties, micro-indentation test is performed. The 0.8 mm diameter ball indenter (SMAC for indentation) would load below 1N to the region between the inserted DMS and host tissue. By this micro loading, the deformation pattern between the DMS inserted sample and PDGF conjugated DMS inserted sample is compared. Successful completion of these studies further validate the present approach and set the stage for further optimization and standardization of the scaffold for testing in a large animal model and preparation for clinical testing.

### Example 9

### Methods and Results

In one embodiment, the inventors examined the potential of PDGF-coated decellularized meniscus scaffold in mediating integrative healing of meniscus tears by inducing endogenous cell migration.

Meniscus Explants:

Fresh bovine menisci (medial and lateral) were obtained from normal knees of 18-30 months old animals (Animal Technologies Inc., Tyler, Tex.). The knees were harvested on the same day that the animals were slaughtered and shipped on ice for arrival in the laboratory the following day. For preparing meniscal explants, the avascular (inner two thirds) was resected with a scalpel and cut into blocks of approximately 20 mm width. The tissue blocks were washed 3 times in DMEM with 1% PSF and incubated in DMEM with 10% calf serum (CS) (Omega Scientific Inc., Tarzana, Calif.) and 1% Penicillin-Streptomycin-Gentamycin (PSG) (Life Technologies) for 3 days.

Preparation of Decellularized Meniscus Scaffold and Growth Factor Conjugation:

Disc shaped explants, 6-mm diameter and 1-mm thickness, were obtained from horizontally punctured bovine meniscus blocks using specimen needles. For decellularization, the explants were sequentially incubated in a shaking incubator (300 rpm at 37° C.) with DNAse/RNAse-free water for 12 hours, 0.05% trypsin-EDTA for 12 hours, washing three times with saline for 1 hour, the mixture of 2% aqueous Triton X-100 and 1.5% peracetic acid for 24 hours, and 2% collagenase for 4 hours. Upon completion of this sequential chemical treatment, decellularized meniscus scaffolds (DMS) were washed with distilled DNAse/RNAse-free water for 72 hours with daily media changes and stored in PBS with 0.1% PSG at 4° C. until use. Analysis of DNA content showed that native meniscus had 7.75 ng/mg tissue dry weight and decellularized DMS 1.71 ng/mg (p=0.0014).

To immobilize PDGF-BB on DMS, heparin was conjugated to the DMS. Heparin sodium salt 0.1% (wt/v) (Sigma Aldrich, St. Louis, Mo.) was dissolved in 0.05M 2-morpholinoethane sulfuric acid (MES) (Sigma-Aldrich) buffer (pH 5.5) containing 25 mM 1-ethyl-3-(3-dimethylaminopropyl) carbodiimide (EDC) (Thermo Scientific) and 10 mM N-hydroxysuccinimide (NHS) (Sigma-Aldrich) to activate the carboxyl groups of heparin. One ml aliquots of the heparin solution were added to each DMS and incubated at room temperature for 4 hours with gentle shaking. The heparin conjugated DMS were rinsed 3 times with 0.1M Na₂HPO₄ and distilled water. The toluidine blue assay showed that the heparin conjugated DMS by EDC cross-linking was 33.77 folds higher than the DMS only immersed in the heparin solution (p<0.0001).

The heparin-conjugated DMS were incubated with recombinant human PDGF-BB (Peprotech Inc., Rocky Hill, N.J.) at a concentration of 200 ng/ml for 12 hours at 4° C. After the PDGF-BB immobilization, DMS were washed 3 times with PBS and stored at 4° C.

Release Kinetics of PDGF-BB from Heparin Conjugated DMS:

Heparin-conjugated DMS (10 mm diameter) was incubated with total amount of 200 ng of PDGF-BB in 1 ml of 0.1% NaN₃ added PBS for 12 hours at 4° C. To remove the non-bound PDGF-BB, the scaffold was washed with 500 μl release media composed of PBS with 0.1% BSA and 0.1% NaN₃. After washing, the supernatant was harvested and the amount of PDGF-BB measured was considered as the non-absorbed PDGF-BB and used to calculate the amount of PDGF-BB bound to the scaffold (total amount of bound PDGF-BB=200 ng—the quantity of non-absorbed PDGF-BB from the rest supernatant). To measure PDGF-BB release from, replicate PDGF-BB-conjugated DMS were placed into 1 ml release media in sterile 1.5 ml tubes at 37° C. with gentle shaking at 150 rpm for up to 16 days. At each time point, all of the media was replaced with fresh release media. The harvested supernatants were centrifuged and stored at −80° C. The cumulative amount of PDGF-BB in the release media from each sample was analyzed using a human PDGF-BB ELISA (Peprotech Inc).

In Vitro Culture of Avascular Cell Seeded on DMS:

One million human avascular meniscus cells (isolated normal knee joints of a 33 year old male and 39 year old female, obtained from tissue banks) were seeded on each DMS (3 mm diameter, 6 mm height) in trans-well plates (12 multi-well plate). After two sequential applications of 50 μl cell suspension onto the surface of DMS, the trans-well plate was incubated at 37° C., 5% CO₂ for 3 hours and then basal culture media or PDGF-BB (50 ng/ml) media was added. The cell-seeded DMS was cultured for 2 weeks with media change every 3 days.

DMS were processed for DAPI and Safranin 0 staining to determine whether cells infiltrate into the DMS and produce new glycosaminoglycan (GAG). To confirm the biological activity of PDGF-BB, anti-PDGFRβ (Ab107169, 1:200 dilution, Abcam) antibody was applied and detected by anti-rabbit IgG (A-11008, 1:500 dilution, Life Technologies).

Ex Vivo Meniscus Explants Culture:

A full thickness radial tear in the middle segment of the meniscus explant was created with a sterile blade. The 6-mm diameter DMS was inserted into the defect. During the insertion, the fiber orientation of the DMS was kept identical to that of the middle part of the meniscus. The explants were cultured for 2 and 4 weeks with media change every 3 days.

Histological and Immunohistochemical Analyses:

The explants (n=5 per each group) were fixed in Z-fix (Anatech, Battle Creek Mich.). Paraffin-embedded sections (5-7 μm) were stained with DAPI (H-1500 Vector Laboratories, Inc.) for cell counting. The percentage of cells at the borderline was calculated with total cells numbers within the explant and compared statistically with control explants. Sections were stained with Safranin-O to detect proteoglycans and with pircrosirius red to detect collagen fiber alignment and interconnectivity between inserted DMS and injured explants. PDGFRβ positive cells were counted to confirm PDGF-BB activity from the conjugated DMS. To confirm the biological activity of PDGF-BB, anti-PDGF receptor beta (Ab107169, 1:200 dilution, Abcam), collagen type 1a1 (Ab34710, 1:500 dilution, Abcam), collagen type 2a1 (II-II6B3, 1:50 dilution, DSHB), and aggrecan (L0101, 1:50 dilution, OWL) antibody were applied and detected by anti-rabbit IgG (A-11008, 1:500 dilution, Life Technologies) or anti-mouse DAB (MP-7420, 1:20 dilution, Vector Laboratories).

Biomechanical Testing:

The Young's modulus of the injured meniscus explants with various types of DMS, including DMS, Heparin-conjugated (HEP) DMS, 50 ng/ml PDGF-BB coated HEP-DMS, 100 ng/ml PDGF coated HEP-DMS, and 200 ng/ml PDGF coated HEP-DMS were quantified by tensile testing (n=8-12 per group). Each explant was mounted and fixed in the grips at their two ends of a uniaxial testing machine (Instron® Universal Testing Machine, 3342 Single Column Model, Norwood, Mass.) with a 500N load cell and tested to failure at a crosshead speed of 1 mm/min at a gauge length of 20 cm under ambient conditions. Young's modulus was calculated from the slope of the linear segment of the stress-strain curve.

Statistical Analysis:

Data represent mean and standard error of mean (SEM), from at least 3 to 4 replicate experiments, each performed in triplicate. The statistical significance of differences in PDGF release quantification was determined using 2-way ANOVA for multiple comparisons. In tensile testing, Mann-Whitney test was used. Differences in histological scores and values, DNA content, and Toluidine blue quantification were analyzed by unpaired t test. Results with *=p<0.05 (95% CI, confidence interval), **=p<0.01 (99% CI), ***=p<0.001 (99.9% CI), ****=p<(99.99% CI) were considered statistically significant.

PDGF Activity and Cell Migration:

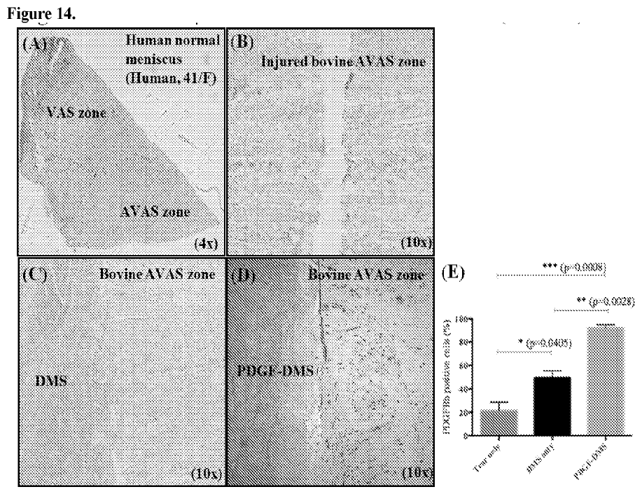

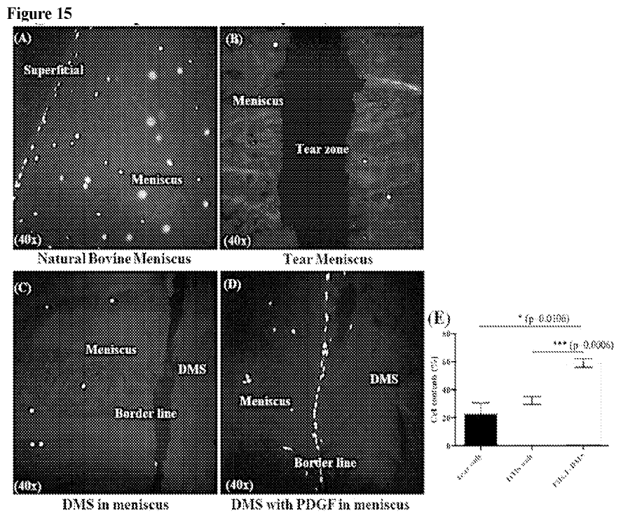

PDGFRβ positive cells in normal meniscus tissue are mainly in the vascular zone (FIG. 14A). PDGF conjugation increased the number of PDGFRβ positive cells among the migrated cells in the defect area (FIG. 14). The PDGF conjugated DMS (92.32±2.536%) was significantly higher positive cells of anti-PDGFRβ than the only DMS inserted group (49.61±5.967%), and only defected meniscus group (21.53±7.267%). Insertion of the PDGF-coated DMS into the meniscus tears led to migration of bovine meniscus cells to the defect zone (FIG. 15). DAPI positive counting revealed PDGF-coated DMS (58.90±3.051%) induced significantly higher cell density to the defect area than the DMS (32.25±2.754%) not conjugated with PDGF.

ECM Formation in the Injured Meniscus Explants:

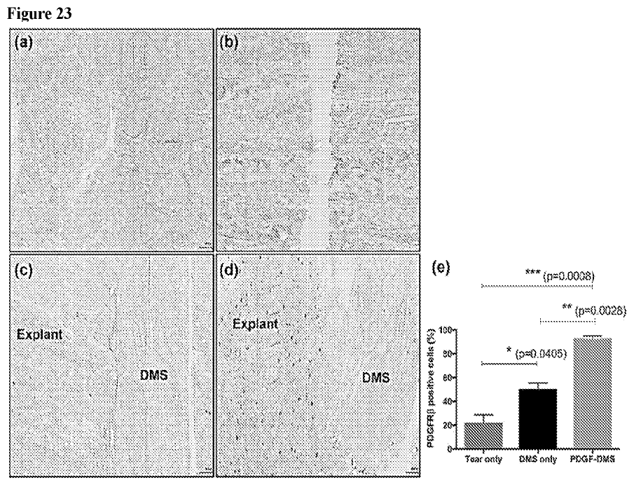

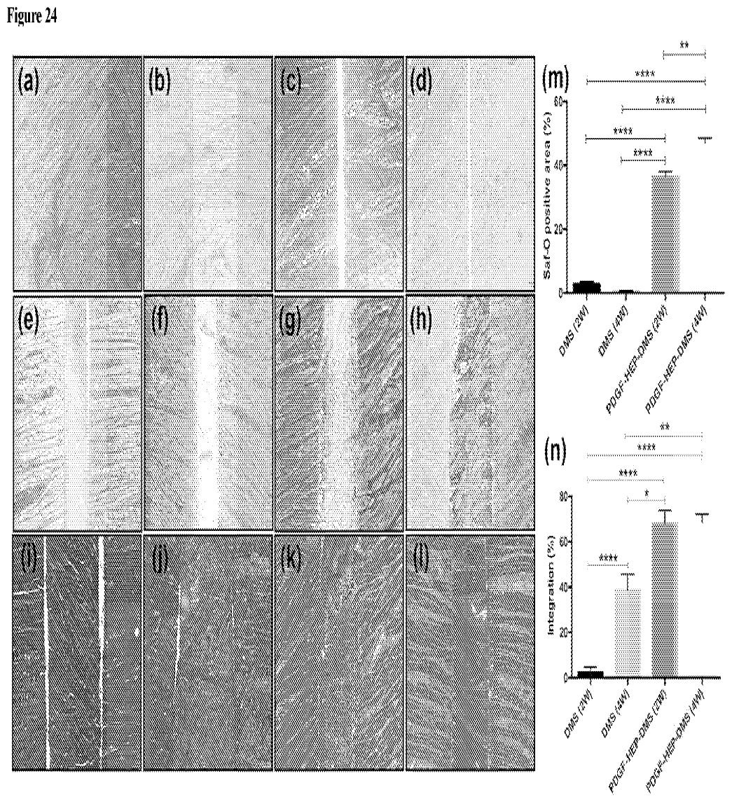

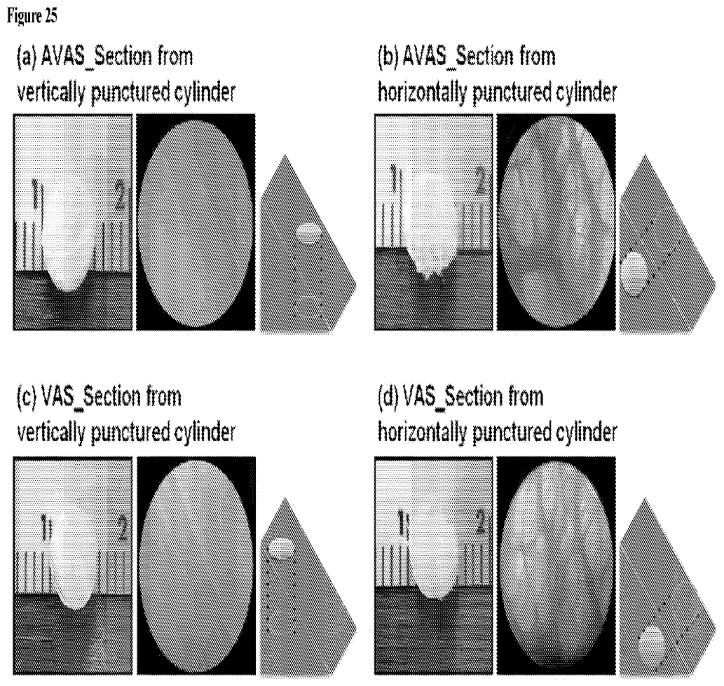

In the histomorphometric analysis of Safranin-O (n=3-9) and picrosirius red (n=4-8) staining, PDGF conjugated DMS induced GAG diffusion into the DMS and tissue integration between DMS and injured explants (FIG. 20). Moreover, the PDGF conjugation promoted cell integration into the DMS. The Safranin-O positive area within the group of PDGF-HEP conjugated DMS (36.49±1.55% after 2 weeks, 46.88±1.673% after 4 weeks) was significantly higher than only DMS inserted meniscus explant (2.69±0.75% after 2 weeks, 0.33±0.30% after 4 weeks). The integration percentage in the PDGF conjugated DMS inserted explants (68.05±5.779% after 2 weeks, 68.45±3.709% after 4 weeks) was significantly higher than only DMS inserted explants (2.313±2.313% after 2 weeks, 38.70±6.981% after 4 weeks)

Mechanical Properties of the Injured Meniscus Explants after DMS Insertion:

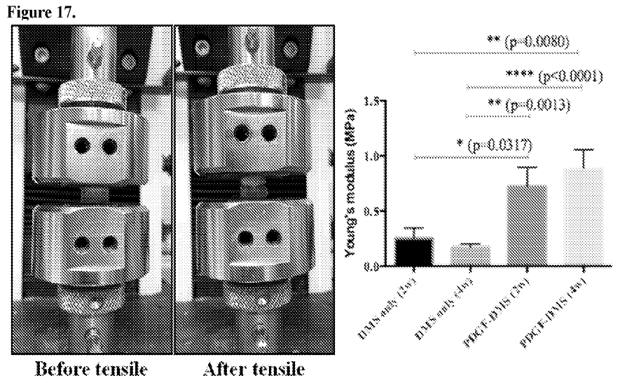

As illustrated in FIG. 17, tensile properties were compared between explants inserted with DMS only or DMS conjugated with 200 ng/ml of PDGF-BB after culture for 2 and 4 weeks. Explants inserted with PDGF-BB-conjugated DMS showed significantly higher Young's moduli (0.73 MPa after 2 weeks, 0.89 MPa after 4 weeks) than only DMS inserted explants (0.25 MPa after 2 weeks, 0.17 MPa after 4 weeks) (FIG. 17).

In conclusion, PDGF coated scaffold increased PDGFRβ expression and promoted migration of endogenous meniscus cells to the defect area and into the scaffold. New matrix was formed that bridged the space between the native meniscus and the scaffold and this was associated with improved biomechanics properties. The PDGF coated scaffold will be promising for translational approach to healing of meniscus tears

### Example 10

### Integrative Healing of Meniscus Tears

In one embodiment, the inventors had previously disclosed that PDGF showed strong chemotactic activity for human articular chondrocytes and bone marrow mesenchymal stem cells (MSC). Y Mishima and M Lotz, J Orthop Res. 2008 October; 26(10):1407-12, the entire disclosure of which is incorporated by reference herein. Even though PDGF is well known as an enhancer of meniscal cell activity, its incorporation into scaffolds should be essential for recruitment of cells to initiate repair in the injured meniscus. The present disclosure is towards a decellularized meniscus sheet for a therapeutic approach to meniscus tears. In one embodiment, the inventors examined the potential of PDGF-coated decellularized meniscus scaffold in mediating integrative healing by endogenous cell migration.

Fresh bovine meniscus was chemically decellularized. Round sheets were made from the decellularized tissue. Heparin was covalently conjugated with decelluarized meniscus scaffold (DMS). PDGF-BB was immobilized by binding to the heparin conjugated DMS. In vitro PDGF release kinetics was analyzed by ELISA. DMS was transplanted into the injured meniscus explants and cultured for 2 and 4 weeks. The numbers migrated cells at the border between DMS and injured explant were counted on DAPI stained sections and PDGFRβ expressing cells were counted after immunohistochemical staining. The newly produced ECM and collagen fiber alignment was detected by histology on Safranin-O and picrosirius red stained sections. The explants were also tested for tensile properties.

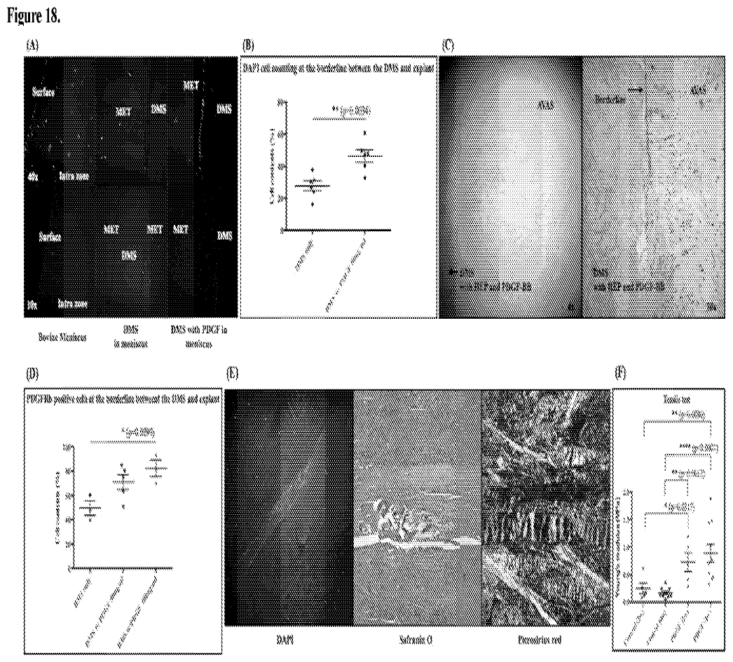

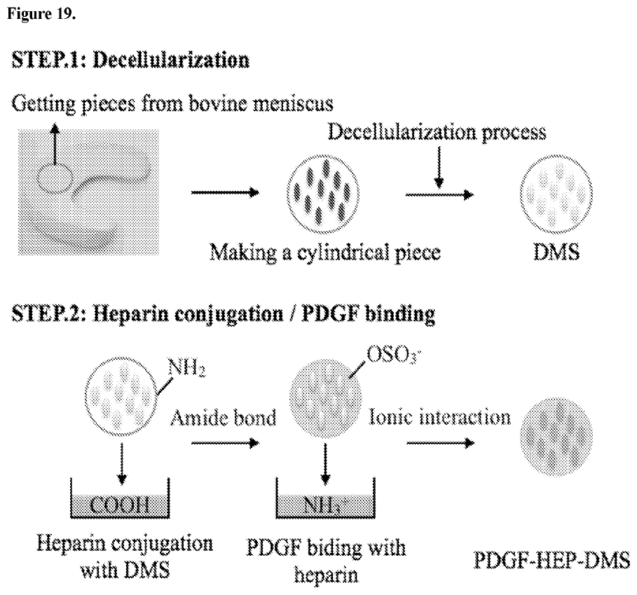

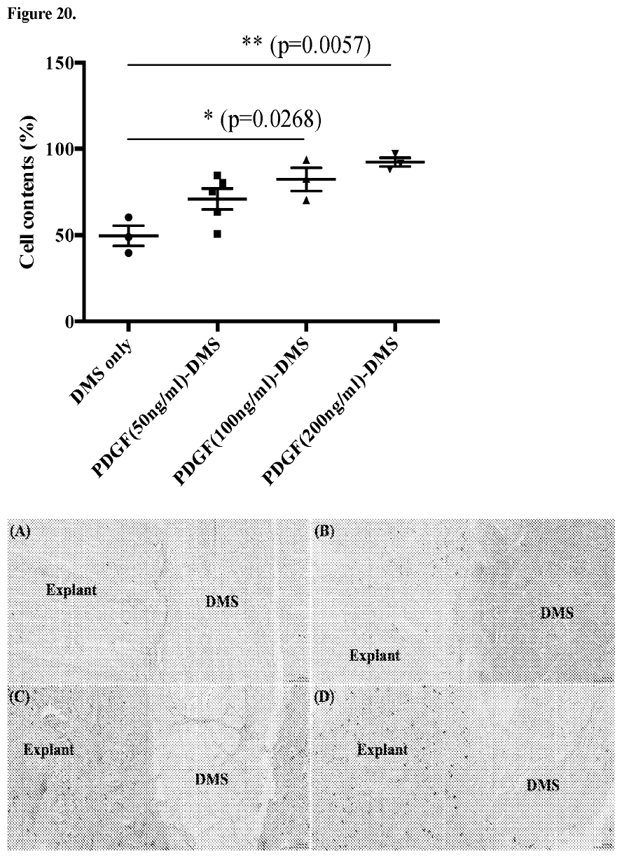

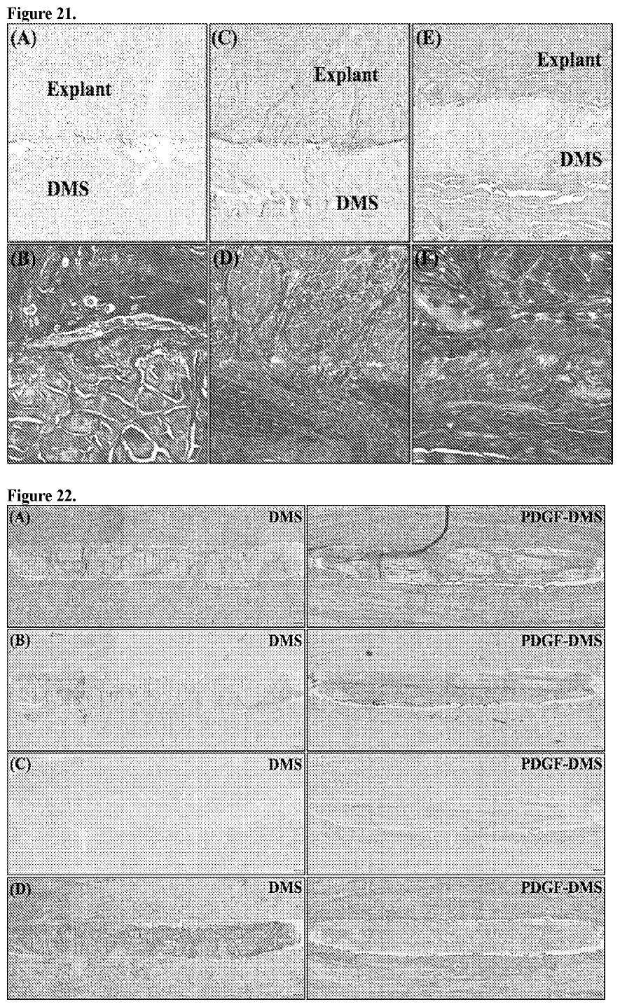

PDGF release kinetics showed sustained slow release in heparin conjugated DMS, with 11.2% release at day-16th compared to 26.1% release from the DMS without heparin. Insertion of the PDGF treated DMS into the meniscus tears led to migration of bovine meniscus cells to the defect zone (FIG. 18 A-D). The migrated cells produced new ECM in the defect area. Safranin-O and pircrosirius red staining showed tissue integration between DMS and injured explants. Moreover, the higher concentration of PDGF promoted cell integration into the DMS (FIG. 18E). Tensile properties of injured explants treated with PDGF coated DMS were significantly higher than in DMS without PDGF (FIG. 18F).

Heparin conjugated DMS showed strong immobilization of PDGF, which was released slowly. PDGF coated DMS promoted migration of endogenous meniscus cells to the defect area and into the scaffold. New matrix was formed that bridged the space between the native meniscus and the scaffold and this was associated with improved biomechanical properties. The PDGF coated DMS is a promising approach for healing of the meniscus tears. In sum, these results provide a novel, feasible and efficient approach for the treatment of meniscus tears.

### Example 11

### Cell Migration and ECM Formation

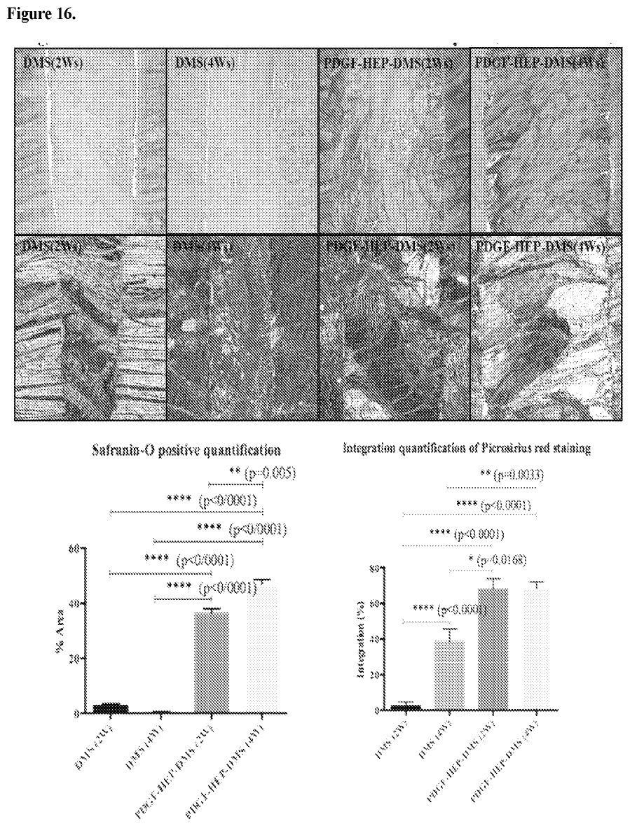

Culture of injured meniscus explants where the defect area was sutured but without insertion of DMS for up to 4 weeks did not show any cell migration or fibrous connectivity in the defect area (FIG. 16). Insertion DMS that was not conjugated with heparin or PDGF also did not show cell migration to the defect area although DMS was filled with the defect area. Insertion of the PDGF treated DMS into the meniscus tears led to migration of bovine meniscus cells to the defect zone (FIG. 16). There was alignment of cells at the border between the meniscus tissue and the inserted DMS. Most of the recruited cells were in the defect space but some cells were migrated into the outer surface of DMS. Cell counting of DAPI stained sections revealed PDGF conjugated DMS (58.90±3.051%) induced significantly higher cell density to the defect area than the DMS (32.25±2.754%) not conjugated with PDGF.

Sections from the same meniscus explants were also stained with PDGFRβ antibody. Results showed that PDGF coated DMS induced a significant increase in the number of PDGFRβ positive cells throughout the explants (FIG. 8). 200 ng/ml of PDGF conjugated DMS (92.32±2.536%) was significantly higher positive cells of anti PDGFRβ among the migrated cells in the defect area than the only DMS inserted group (49.61±5.967%), and only defected meniscus group (21.53±7.267%). This biological activity of PDGF coated DMS was confirmed by PCR on RNA isolated form the cultured explants.

ECM formation in the injured meniscus explants was studied by Safranin-O and pircrosirius red staining. Safranin-O and pircrosirius red staining showed tissue integration between DMS and injured explants. Moreover, the higher concentration of PDGF promoted cell integration into the DMS.

Mechanical properties of the injured meniscus explants were also studied. Tensile property was compared between only DMS inserted explants and 200 ng/ml of PDGF conjugated DMS inserted explants after 2 and 4 weeks. PDGF conjugated DMS inserted explant (0.73 MPa after 2 weeks, 0.89 MPa after 4 weeks) at each time point was significantly higher than only DMS inserted explant (0.25 MPa after 2 weeks, 0.17 MPa after 4 weeks).

In one embodiment, PDGF-BB binding with heparin conjugated DMS increased PRGFRβ expression in the avascular zone. This increase made cell migration near to the PDGF-BB binding DMS in the meniscus defect. PDGF/PDGFR signaling studies showed the interaction in the endothelial progenitor cells, or mesenchymal stem cells. There also had been limitations about regional variation in response to PDGF-BB, which was critical in the avascular region in vitro. This is the first study that PDGF-BB immobilized DMS can induce the increase of PDGFRβ expression and that lead to cell migration and proliferation near to the meniscal defect region in native tissue. In one embodiment, at the early point after PDGF-BB treatment, the VEGFA expression was increased.

In one embodiment, the data and results disclosed herein show that heparin conjugated DMS showed strong immobilization of PDGF, which was released slowly. PDGF coated DMS promoted migration of endogenous meniscus cells to the defect area and into the scaffold. New matrix was formed that bridged the space between the native meniscus and the scaffold and this was associated with improved biomechanical properties. The PDGF coated DMS is a novel and promising approach for healing of the meniscus tears.

### Example 12

### Cell Migration and ECM Formation in DMS-Inserted Meniscus Tears

Culture of injured meniscus explants where the defect area was sutured but without insertion of DMS for up to 2 weeks did not show any cell migration or fibrous connectivity in the defect area (FIG. 15b). Insertion of DMS that was not conjugated with heparin or PDGF also did not show cell migration to the defect area although the defect space was filled with DMS (FIG. 15c).

Insertion of the PDGF-BB-conjugated DMS into the meniscus tears led to migration of meniscus cells to the defect zone (FIG. 15d). There was alignment of cells at the border between the meniscus tissue and the inserted DMS. Most of the recruited cells were in the defect space but some cells migrated into the outer surface of the DMS.

Cell counting of DAPI stained sections revealed that PDGF-BB-conjugated DMS (58.90±3.051%) induced significantly higher cell density in the defect area than the DMS (32.25±2.754%) not conjugated with PDGF.

Sections from the same meniscus explants were also stained with PDGFRβ antibody. PDGFRβ expression was seen in most cells in human meniscus in the vascular zone. However exogenous PDGF-BB induced PDGFRβ expression in the avascular zone. Moreover, endogenous PDGFRβ gene expression was increased by PDGF-BB treatment in the avascular zone. In cultured bovine meniscus, PDGF-BB-coated DMS induced a significant increase in the number of PDGFRβ positive cells throughout the explants (FIG. 15d). PDGF-BB-conjugated DMS (92.32±2.536%) induced significantly higher numbers of PDGFR-positive cells among the migrated cells in the defect area than the DMS inserted group (49.61±5.967%), and the sutured meniscus group without DMS (21.53±7.267%) (FIG. 15e).

### Example 13

### ECM Formation in the Injured Meniscus Explants

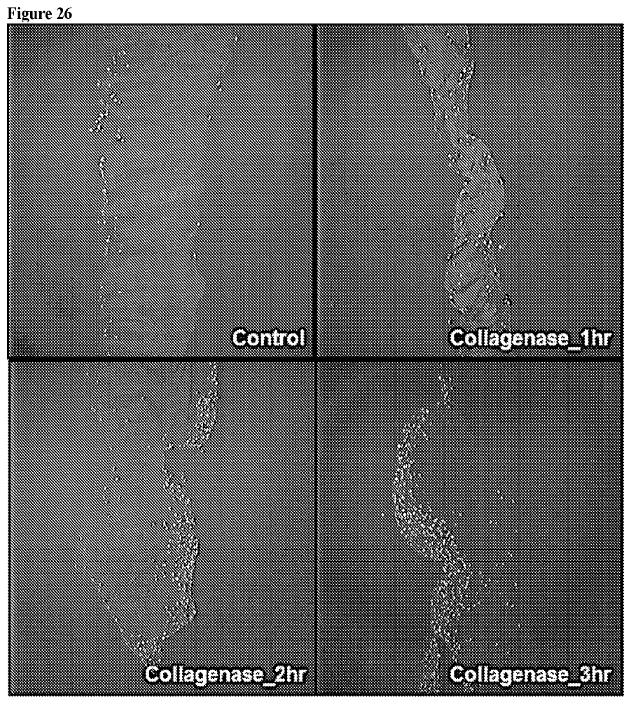

Safranin-O and picrosirius red staining showed tissue integration between DMS and injured explants (FIG. 24a-1). The Safranin-O positive area assessed by image analysis in PDGF-BB-conjugated DMS group after 2 weeks (34.491±1.55%) and 4 weeks-culture (46.88±1.673%) was significantly higher than in the DMS group after 2 weeks (2.69±0.75%) and 4 weeks-culture (0.33±0.31%) (FIG. 24m). The interconnectivity area between the inserted DMS and injured bovine meniscus explant in PDGF-BB-conjugated DMS group after 2 weeks (68.1±5.78%) and 4 weeks-culture (68.45±3.71%) was significantly higher than in the DMS group after 2 weeks (2.31±2.31%) and 4 weeks-culture (38.7±6.98%) (FIG. 24n).

### Example 14

### Growth Factor Conjugated Scaffold

In various embodiments disclosed herein, the present disclosure provides a growth factor-conjugated scaffold that can be readily applied in the clinic to recruit endogenous cells that promote repair of meniscus tears. In one embodiment, DMS was selected as the scaffold because it is a biocompatible material and can be readily manufactured for clinical use.

Prior studies about growth factor-conjugated scaffolds including natural polymers such as collagen, gelatin, demineralized bone matrix, and synthetic polymer have shown the feasibility of growth factor immobilization for cell recruitment and tissue repair. Scaffolds for meniscus repair in clinical applications need not only to promote endogenous cell recruitment but also have mechanical properties to resist shear and compressive stresses in the knee joint.

DMS has been used as a scaffold, having similar mechanical properties as human meniscus but there is no study about growth factor immobilized DMS for endogenous cell recruitment in meniscus. During decellularization the dense bovine meniscus was modified by proteolytic enzyme treatment to facilitate subsequent cell infiltration.

PDGF-conjugated DMS was biologically active after insertion into meniscus explants as demonstrated by increased PRGFRβ expression on cells in the avascular zone. This was associated with cell migration to the PDGF-BB-conjugated DMS in the meniscus defect. Previous studies showed that PDGF/PDGFR signaling is involved on defining phenotype and regulating function of endothelial progenitor cells, or mesenchymal stem cells. PDGFβ receptor positive cells have been reported to include or represent stem/progenitor populations and the present results indicate that these cell populations in meniscus are recruited and/or activated by PDGF. Multipotent meniscus progenitor cells are more migratory in OA or diseased meniscus than healthy meniscus.

The present disclosure is believed to be the first to show that by PDGF-BB immobilized DMS stimulates cell migration into the meniscal defect. The recruited cells also produced new extracellular matrix and increased interconnectivity between the PDGF coated DMS and defect region with newly released ECM. PDGF is not only chemotactic but also enhances synthesis of fibrocartilage matrix components such as GAG and collagens. The enhanced interconnectivity by the new ECM increased biomechanical property such as initial tensile Young's modulus.

At the early time points after PDGF-BB treatment, the PDGFRβ and VEGFA gene expressions were increased in avascular meniscus tissue and cells (FIGS. S6 and 7). The VEGF-mediated neovascularization is essential to the healing of injured tissues. In cultured meniscal cells, VEGF in vascular meniscal cells was higher than in avascular meniscal cells. Also, VEGF was detected mainly around injured areas of the meniscus. VEGF expression in avascular cells by PDGF-BB treatment may modulate the meniscus healing process in the avascular zone.

Studies about insertion of membranes into the experimental meniscus tears have been reported. The insertion of collagenase-releasing nanofibrous scaffold showed enhancement of the cell infiltration by loosening the dense meniscus explant. However, there was no integration at the edge occupied by the scaffold at early time point. A multi laminated-collagenous biomaterial was conductive for cell repopulation with host meniscal elements but studies are necessary to show sufficient mechanical properties.

### Example 15

### DMS Optimization by Orientation

As illustrated in FIG. 25, in one embodiment, DMS optimization was done to align the collagen fiber orientation of the DMS with the collagen orientation in the meniscus defect. The bovine meniscus was separated from vascular (FIG. 25c, 25d) and avascular (FIG. 25a, 25b) regions. Using a 6-mm specimen needle, cylindrical tissue pieces were collected in vertical direction (FIG. 25a, 25c) or horizontal direction (FIG. 25b, 25d). Then, 1-mm thickness disc shaped scaffold was sliced from each piece and decellularized. After the DMS preparation, the microscopic structure showed significantly different morphology between vertical and horizontal sections. The horizontally punctured tissue pieces showed inner-fibrous network for better cell migration and infiltration.

### Example 16

### DMS Optimization by Creating Additional Pores

As illustrated in FIG. 26, further optimization of DMS was done to create pores in the dense collagen matrix of the decellularized meniscus scaffold to facilitate cell migration and infiltration. The DMS scaffold contains dense collagen fiber network. To facilitate cell migration and infiltration, pores were created by using collagenase digestion, mechanical puncture, or laser application. FIG. 26 illustrates the effect of collagenase digestion of DMS on cell infiltration. Short treatment with collagenase was chosen (2% wt/v for 3-4 hours). 2M synovial mesenchymal stem cells were cultured on each DMS 6-mm diameter disc, which was followed by DAPI staining (10×). The results showed increased cell infiltration after a short treatment with collagenase (FIG. 26).

### Example 17

### PDGF-Conjugated DMS Induces Cell Migration and Proliferation

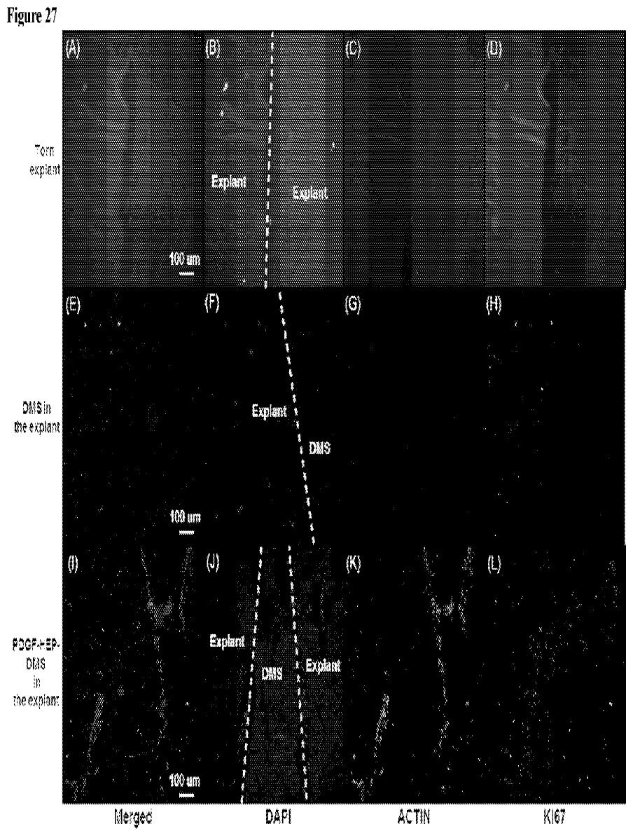

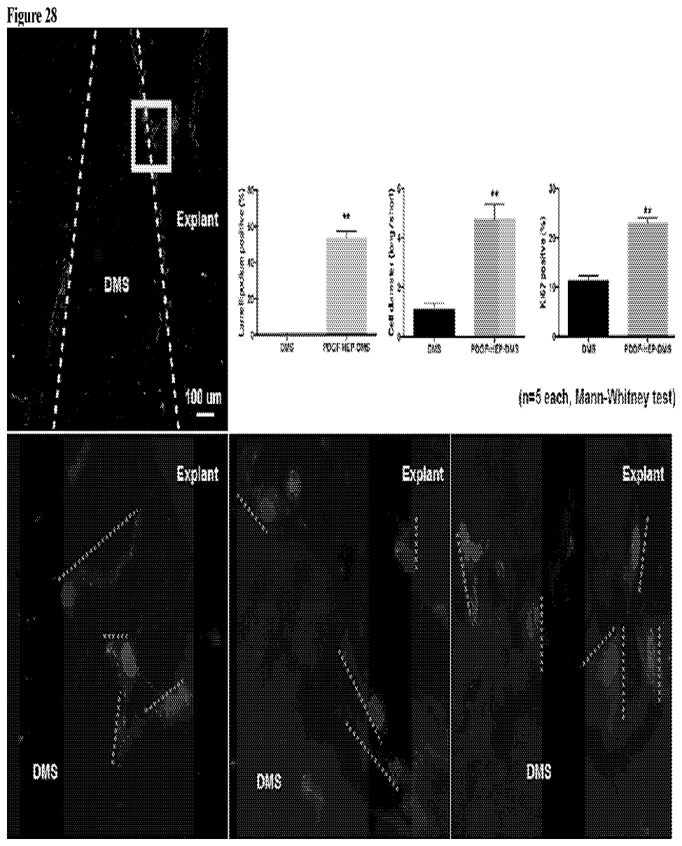

As illustrated in FIGS. 27 and 28, PDGF-conjugated DMS induces cell migration and proliferation. PDGF conjugated DMS was inserted into experimental defect in bovine meniscus explant. After 2 week-ex vivo culture, immuno-fluorescence analysis was performed for cell migration and proliferation. Positive actin staining (red), represents lamellipodia and indicates direction of the cell migration. KI67 (green, FIG. 27) positive staining indicates proliferative cells. These results demonstrate that PDGF conjugated DMS induces more cell migration and proliferation (100× confocal image)

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps, some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a,” “an,” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A scaffold comprising:
a decellularized meniscus tissue,
wherein the scaffold is covalently conjugated with heparin and a growth factor.

2. The scaffold of claim 1, wherein the growth factor is selected from the group consisting of Platelet-derived growth factor (PDGF), Transforming growth factor beta (TGFβ), Vascular endothelial growth factor (VEGF), Connective tissue growth factor (CTGF), Fibroblast growth factor (FGF), and other chemokines such as CCL20, CXCL3, CXCL6, CCL3, CCL3L1.

3. The scaffold of claim 1, wherein the growth factor is platelet derived growth factor (PDGF).

4. The scaffold of claim 3, wherein the PDGF is PDGF-AA, PDGF-BB, and/or PDGF-AB.

5. The scaffold of claim 1, wherein the scaffold further comprises stem cells.

6. The scaffold of claim 1, wherein the scaffold further comprises meniscus cells.

7. The scaffold of claim 1, wherein the decellularized meniscus tissue comprises collagen fibers, and wherein the collagen fiber orientation is matched with that of a meniscus defect.

8. The scaffold of claim 1, wherein the decellularized meniscus tissue comprises pores.

9. The scaffold of claim 8, wherein the pores are created in the decellularized meniscus tissue by collagenase digestion, mechanical puncture, and/or laser application.

10. The scaffold of claim 1, wherein the scaffold releases the growth factor with substantially first order kinetics over a period of at least 10 days after administration.

11. The scaffold of claim 1, wherein the scaffold releases the growth factor with substantially first order kinetics over a period of at least 20 days after administration.

12. The scaffold of claim 1, wherein the scaffold releases the growth factor with substantially first order kinetics over a period of at least 30 days after administration.

13. The scaffold of claim 1, wherein the tensile strength of the scaffold is at least two times greater than a tensile strength of a similar decellularized meniscus tissue, but without covalent conjugation of heparin and the growth factor.

14. The scaffold of claim 1, wherein the tensile strength of the scaffold is at least three times greater than a tensile strength of a similar decellularized meniscus tissue, but without covalent conjugation of heparin and the growth factor.

15. The scaffold of claim 1, wherein the tensile modulus of the scaffold is greater than 0.6 Young's Modulus (MPa).

16. The scaffold of claim 1, wherein the growth factor comprises between 10 ng/mL to 1 mg/mL of the scaffold.

17. The scaffold of claim 1, wherein the decellularized meniscus tissue is essentially in a sheet form.

18. The scaffold of claim 1, wherein the decellularized meniscus tissue has a three dimensional form.

19. The scaffold of claim 1, wherein the scaffold is in a medical dressing.

20. The scaffold of claim 1, wherein the decellularized meniscus tissue originates from a mammal.

21. The scaffold of claim 1, wherein the decellularized meniscus tissue originates from a human.

22. The scaffold of claim 1, wherein the scaffold is in a sterile condition and packaged in a sterile container.

23. A method of repairing and/or treating a tissue injury in a subject in need thereof, comprising:
providing a scaffold comprising a decellularized meniscus tissue; and
repairing and/or treating the tissue injury by implanting the scaffold over the tear,
wherein the scaffold is covalently conjugated with heparin and a growth factor.

24. The method of claim 23, wherein the tissue injury is a tear in the tissue.

25. The method of claim 23, wherein the tissue is a meniscus tissue.

26. The method of claim 23, wherein the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1.

27. The method of claim 23, wherein the growth factor is platelet derived growth factor (PDGF).

28. The method of claim 27, wherein the PDGF is PDGF-AA, PDGF-BB, and/or PDGF-AB.

29. The method of claim 23, wherein the scaffold recruits new population of cells to initiate repair in the avascular zone of meniscus tissue.

30. The method of claim 23, wherein the scaffold is optimized for effective cell infiltration and migration from host cells to the scaffold.

31. The method of claim 23, wherein the acellular scaffold is implanted over the meniscus tear by an arthroscopic surgery.

32. The method of claim 23, wherein the scaffold releases the growth factor with substantially first order kinetics over a period of at least 10 days after administration.

33. The method of claim 23, wherein the scaffold releases the growth factor with substantially first order kinetics over a period of at least 20 days after administration.

34. The method of claim 23, wherein the scaffold releases the growth factor with substantially first order kinetics over a period of at least 30 days after administration.

35. The method of claim 23, wherein the tensile strength of the scaffold is at least two times greater than a tensile strength of a similar decellularized meniscus tissue, but without covalent conjugation of heparin and PDGF.

36. The method of claim 23, wherein the tensile strength of the scaffold is at least three times greater than a tensile strength of a similar decellularized meniscus tissue, but without covalent conjugation of heparin and PDGF.

37. The method of claim 23, wherein the tensile modulus of the scaffold is greater than 0.6 Young's Modulus (MPa).

38. The method of claim 23, wherein PDGF comprises between 10 ng/ml to 1 mg/ml of the scaffold.

39. The method of claim 23, wherein the method of repairing and/or treating the tear in the tissue further comprises a second treatment regimen.

40. The method of claim 39, wherein the second treatment regimen comprises a non-surgical treatment, such as rest, ice, compression, elevation, and/or physical therapy.

41. The method of claim 39, wherein the second treatment regimen comprises a surgical treatment such as surgical repair, partial meniscectomy, and/or total meniscectomy.

42. The method of claim 23, wherein the subject is a mammal.

43. The method of claim 23, wherein the subject is a human.

44. A kit comprising:
a sterile container comprising a scaffold covalently conjugated with heparin and a growth factor; and
instructions for using the kit.

45. The kit of claim 44, wherein the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1.

46. The kit of claim 44, wherein the growth factor is platelet derived growth factor (PDGF).

47. The kit of claim 44, wherein the PDGF is PDGF-AA, PDGF-BB, and/or PDGF-AB.

48. The kit of claim 44, wherein the kit further comprises a means for delivery of the scaffold into an injured meniscus.

49. The kit of claim 48, wherein the means of delivery is medical glue, medical sutures, medical staples, and/or medical anchors.

50. The kit of claim 44, wherein the scaffold is a biological acellular scaffold.

51. The kit of claim 44, wherein the scaffold is derived from decellularized native meniscus tissue.

52. The kit of claim 44, wherein the acellular scaffold recruits new population of cells to initiate repair in the avascular zone.

53. The kit of claim 44, wherein the heparin conjugation enables slow release of the growth factor.

54. The kit of claim 44, wherein the slow release occurs over a period of up to 30 days.

55. A device comprising:
an acellular scaffold covalently conjugated with heparin and a growth factor,
wherein the device is for repairing tissues.

56. The device of claim 55, wherein the growth factor is selected from the group consisting of PDGF (Platelet-derived growth factor), TGFβ (Transforming growth factor beta), VEGF (Vascular endothelial growth factor), CTGF (Connective tissue growth factor), FGF (Fibroblast growth factor), and other chemokines such as CCL20, CXCL3, CXCL6, CCL3, CCL3L1.

57. The device of claim 55, wherein the growth factor is platelet derived growth factor (PDGF).

58. The device of claim 55, wherein the acellular scaffold is a biological acellular scaffold.

59. The device of claim 55, wherein the acellular scaffold is derived from decellularized native meniscus tissue.

60. The device of claim 55, wherein the acellular scaffold has similar biological and mechanical characteristics compared with native meniscus.

61. The device of claim 55, wherein the growth factor recruits new population of cells to initiate repair in the avascular zone.

62. The device of claim 55, wherein the acellular scaffold is optimized for effective cell infiltration and migration from host cells to the scaffold.

63. The device of claim 55, wherein the device enables slow release of the growth factor.

64. The device of claim 55, wherein the slow release occurs over a period of up to 30 days.

65. A method of inducing cell migration, comprising:
providing a decellularized meniscus scaffold for the immobilization of one or more growth factors; and
inducing cell migration to the decellularized meniscus scaffold.

66. The method of claim 65, wherein the one or more growth factors is PDGF.

67. The method of claim 65, wherein heparin is used for immobilization.

68. The method of claim 65, wherein the decellularized meniscus scaffold is implanted directly to a subject.

69. The method of claim 65, wherein the subject is a mammal.

70. The method of claim 65, wherein the subject is human.
